(19) United States
(12) Patent Application Publication
(10) Pub. No.: US 2026/0284418 A1
(43) Pub. Date: Sep. 24, 2026

(54) MEDICAL DEVICE FIXATION INTEGRITY MONITORING AND ALERT

(71) Applicant: Medtronic, Inc., Minneapolis, MN (US)

(72) Inventors: Michael P. CAMPBELL, Blaine, MN (US); Kevin T. O'BRIEN, Minneapolis, MN (US)

(21) Appl. No.: 19/469,851

(22) PCT Filed: Mar. 12, 2024
(86) PCT No.: PCT/IB2024/052392

§ 371 Date: Sep. 26, 2025

Related U.S. Application Data
(60) Provisional application No. 63/493,672, filed on Mar. 31, 2023.

(51) Int. Cl. A61N 1/375; A61N 1/365

(52) U.S. Cl. CPC A61N 1/37518; A61N 1/36585

(57) ABSTRACT

An implantable medical device (IMD) is configured to sense an axis signal along a plurality of axes of a motion sensor of the IMD. The IMD is configured to, for multiple time periods determine a metric from the axis signals. Each of the multiple time periods extend over multiple cardiac cycles. The IMD may determine that the metrics determined for at least a plurality of the multiple time periods meet a dislodgement threshold and detect dislodgement of the IMD in response to the metrics determined for at least the plurality of multiple time periods meeting the dislodgement threshold.

This application claims the benefit of U.S. Provisional Patent Application Ser. No. 63/493,672, filed Mar. 31, 2023, the entire content of which is incorporated herein by reference.

### TECHNICAL FIELD

This disclosure relates to an implantable medical device and a method for monitoring the fixation integrity of the implantable medical device.

### BACKGROUND

Medical devices may sense physiological signals from the heart, brain, nerve, muscle or other tissue. Such devices may be implantable, partially implantable, wearable or external devices having sensors for sensing the physiological signals for monitoring a patient condition. In some cases, such devices may be configured to deliver a therapy based on the sensed physiological signals. For example, implantable or external cardiac pacemakers, cardioverter defibrillators, cardiac monitors and the like, sense cardiac electrical signals from a patient's heart. A cardiac pacemaker or cardioverter defibrillator may sense cardiac electrical signals from the heart and deliver electrical stimulation therapies to the heart using electrodes carried by a transvenous medical electrical lead, a non-transvenous medical electrical lead and/or leadless electrodes coupled directly to the housing of the medical device.

The electrical stimulation therapies may include signals such as pacing pulses. In some cases, a medical device may sense cardiac event signals attendant to the intrinsic or pacing-evoked depolarizations of the heart and control delivery of stimulation signals to the heart based on sensed cardiac event signals. Upon detection of an abnormal heart rhythm based on the sensed cardiac event signals (or absence thereof), an appropriate electrical stimulation signal or signals may be delivered to the patient's heart to restore or maintain a more normal heart rhythm.

Intracardiac pacemakers have been introduced or proposed for implantation entirely within a patient's heart eliminating the need for transvenous leads. For example, an intracardiac pacemaker may provide sensing and pacing from within a heart chamber of a patient having a conduction abnormality to promote a more normal heart rhythm. The intracardiac pacemaker may be fixedly positioned at an implant site by a fixation member to hold electrodes of the pacemaker at a desired location for sensing cardiac signals and delivering cardiac pacing pulses.

### SUMMARY

The techniques of this disclosure generally relate to an implantable medical device (IMD) having at least one fixation member for stably anchoring the IMD at an implant site. The IMD may include a motion sensor for sensing a signal responsive to motion of the IMD, e.g., an accelerometer for sensing acceleration of the IMD due to gravity, blood flow, patient body motion or other forces imparted on the IMD. The IMD may be configured to sense other physiological signals, such as electrophysiological signals, impedance signals, etc., and may be configured to deliver a therapy, e.g., cardiac pacing or other electrical stimulation therapy in some examples.

An IMD operating according to the techniques disclosed herein is configured to monitor an acceleration signal produced by an accelerometer included in the IMD for detecting partial or total dislodgement of the IMD from an implant position. Partial or total dislodgement of the IMD may occur due to migration of the IMD or fixation member or degradation of the fixation forces provided by the fixation member. The IMD can be configured to identify changes in the acceleration signal indicative of IMD dislodgement and determine when such changes meet dislodgement criteria. The IMD may be configured to respond to detecting IMD dislodgement when the dislodgement criteria are met by at least generating an alert that can be transmitted to an external device.

In one example, the disclosure provides an IMD including a motion sensor, a sensing circuit, a pulse generator and a control circuit. The motion sensor is configured to sense an axis signal along a plurality of axes of the motion sensor. The sensing circuit is configured to sense cardiac event signals attendant to myocardial depolarizations, and the pulse generator is configured to generate cardiac pacing pulses. The control circuit can be in communication with the motion sensor, the sensing circuit and the pulse generator. The control circuit may be configured to determine a signal from the axis signals over multiple time periods that each extend over multiple cardiac cycles. For each of the multiple time periods, the control circuit may be configured to determine a metric from the signal. The control circuit may be configured to determine that the metrics for at least a plurality of the multiple time periods meet a dislodgement threshold. The control circuit may detect dislodgement of the IMD in response to the metrics determined for at least the plurality of the successive time periods meeting the dislodgement threshold. The IMD may include a communication circuit configured to transmit an IMD dislodgement alert in response to the control circuit detecting the dislodgement.

In another example, the disclosure provides a method including sensing an axis signal along a plurality of axes of a motion sensor of an IMD. The method includes determining a signal from the axis signals sensed over multiple time periods, the multiple time periods each extending over a plurality of cardiac cycles. The method further includes, for each of the multiple time periods, determining a metric from the signal and determining that the metrics determined for at least a plurality of the multiple time periods meet a dislodgement threshold. The method may further include detecting dislodgement of the IMD in response to the metrics determined for at least a plurality of the multiple time periods meeting the dislodgement threshold. The method may include transmitting an IMD dislodgement alert in response to detecting the dislodgement.

In another example, the disclosure provides a non-transitory, computer-readable storage medium comprising a set of instructions which, when executed by a control circuit of an IMD, cause the IMD to sense an axis signal along a plurality of axes of a motion sensor of the IMD and determine a signal from the axis signals over multiple time periods each extending over a plurality of cardiac cycles. The instructions further cause the IMD to determine a metric from the signal for each of the multiple time periods. The instructions may further cause the IMD to determine that the metrics for at least a plurality of the multiple time periods meet a dislodgement threshold and detect dislodgement of the IMD in response to the metrics determined for at least the plurality of the multiple time periods meeting the dislodgement threshold. The instructions may further cause the IMD to transmit an IMD dislodgement alert in response to detecting the dislodgement.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device (IMD) system that may be configured to detect IMD dislodgement according to the techniques disclosed herein.

FIG. 2 is a conceptual diagram of the IMD shown in FIG. 1 having a helical fixation member according to some examples.

FIG. 3 is a conceptual diagram of an IMD having a fixation member provided as one or more fixation tines.

FIG. 4 is a conceptual diagram of a configuration of an IMD according to some examples.

FIG. 5 is a flow chart of a method for detecting IMD dislodgement according to some examples.

FIG. 6 is a diagram of accelerometer axis signals obtained at two different time points.

FIG. 7 is a flow chart of a method for detecting IMD dislodgement based on orientation change metrics according to some examples.

FIG. 8 is a diagram of a plot of long term trend values of an orientation change metric (left side of graph) and new, recent orientation change metrics determined at scheduled monitoring intervals (right side of graph).

FIG. 9 is a flow chart of another method for detecting IMD dislodgement according to some examples.

FIG. 10 is a flow chart of yet another method for detecting IMD dislodgement according to some examples.

FIG. 11 is a flow chart of yet another method for detecting IMD dislodgement according to some examples.

### DETAILED DESCRIPTION

In general, this disclosure describes a medical device and method for detecting at least partial dislodgement of the IMD from an implant position associated with degradation of the fixation integrity of a fixation member of the IMD. As used herein, the term “dislodgement” refers to a change in the position of a fixation member anchoring the medical device at an implant site resulting in change in the position of the medical device relative to the body tissue or anatomical structure(s) at the implant site. As such, dislodgement may include a lateral, angular, or rotational shift of the medical device relative to a previous position of the medical device due to movement of a fixation member of the medical device from a first position relative to the tissue that the fixation member is positioned in. The dislodgement may occur when a fixation member or portion thereof anchoring the medical device at the implant site retracts from (or in some instances advances further into) the tissue at the implant site. The dislodgement may occur when the fixation member or portion thereof anchoring the medical device loses structural integrity, e.g., fractures, partially or completely, or bends beyond an elastic limit. The mode of dislodgement may depend on the type of fixation member and its anchoring mechanism. For example, a fixation member may include multiple fixation tines and dislodgement may occur when one or more tines of the fixation member fractures or retracts from the body tissue, partially or wholly. In another example, a fixation member may be fixation helix that may rotate out of the body tissue, partially or wholly, causing dislodgement of the IMD.

An IMD operating according to the methods disclosed herein is configured to detect a dislodgement of the IMD by monitoring a motion signal produced by a motion sensor included in the IMD. In the illustrative examples presented herein, the medical device includes an accelerometer as the motion sensor. The accelerometer is configured to sense an acceleration signal responsive to acceleration forces imposed on the medical device, e.g., caused by body tissue motion, flowing blood, and/or patient physical activity. However, other types of sensors capable of sensing a signal that is responsive to movement of the medical device could be implemented for use according to the techniques disclosed herein. For example, a gyroscope may be used for sensing IMD motion for detecting dislodgement.

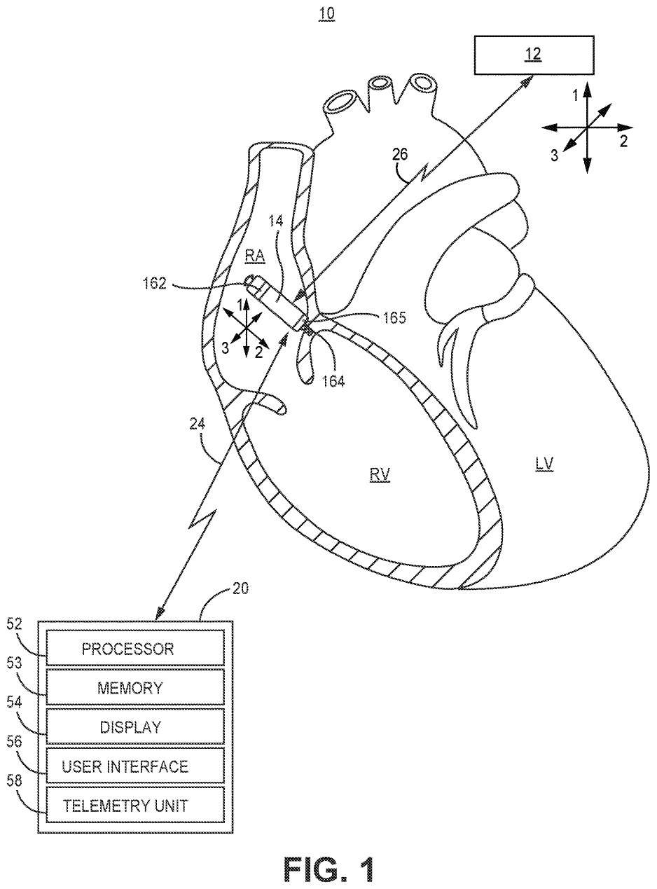

FIG. 1 is a conceptual diagram illustrating an IMD system 10 that may be configured to detect IMD dislodgement according to the techniques disclosed herein. The IMD system 10 includes an IMD 14, which is a cardiac pacemaker in this example, having a fixation member for anchoring the IMD at an implant site. IMD 14 can be a transcatheter, leadless pacemaker that can be implanted wholly within a heart chamber. In the example shown IMD 14 is implanted in the right atrial (RA) chamber of a patient's heart 8. However, IMD 14 may be adapted for implantation in or on an atrial or ventricular chamber of heart 8 in various examples. IMD 14 may be reduced in size compared to subcutaneously implanted pacemakers and may be generally cylindrical in shape to facilitate transvenous implantation via a delivery catheter. IMD 14 can be a leadless pacemaker that includes electrodes carried on the IMD housing without requiring medical electrical leads extending from IMD 14 for sensing cardiac electrical signals and delivering cardiac pacing pulses.

IMD 14 may be capable of sensing atrial and ventricular event signals, e.g., P-waves attendant to atrial depolarizations and R-waves attendant to ventricular depolarizations. IMD 14 may be configured as a dual chamber pacemaker capable of sensing both atrial and ventricular event signals and delivering atrial pacing pulses and ventricular pacing pulses as needed based on the sensed atrial and/or ventricular event signals. In other examples, IMD 14 may be configured as a single chamber pacemaker capable of delivering only atrial pacing pulses or capable of delivering only ventricular pacing pulses but may still be capable of dual chamber sensing of both atrial and ventricular event signals. In still other examples, IMD 14 may be configured to sense and pace a single heart chamber, atrial or ventricular.

In the example shown, IMD 14 is implanted in the RA for providing ventricular pacing from an atrial location. IMD 14 may be configured to deliver ventricular pacing pulses via the heart's native conduction system and/or ventricular myocardium from a RA approach. For example, the distal end of IMD 14 may be positioned at the inferior end of the interatrial septum, beneath the AV node and near the tricuspid valve annulus to position a tip electrode 164, generally in the Triangle of Koch, for advancement into the interatrial septum toward the His bundle of the native His-Purkinje conduction system. As described below, tip electrode 164 may serve as a fixation member for engaging with cardiac tissue for anchoring IMD 14 at the implant site. A second electrode, e.g., a ring electrode 162 or ring electrode 165, may be spaced proximally from the tip electrode 164 for use with the tip electrode 164 for bipolar pacing of the right and left ventricles via the His-Purkinje system and/or ventricular myocardium. Ventricular pacing pulses delivered by IMD 14 may capture at least a portion of the His bundle (or more generally the cardiac conduction system) and/or ventricular myocardium for delivering ventricular pacing from an atrial implant location of IMD 14. The techniques disclosed herein are not necessarily limited to a particular implant location of IMD 14, however, and may be practiced in an IMD implanted in a variety of operative locations for providing physiological signal sensing and/or therapy delivery.

As described below, IMD 14 may include an accelerometer configured to sense acceleration forces imparted on IMD 14. The accelerometer may be three dimensional accelerometer configured to produce an acceleration signal along each of three axes, which may be orthogonal axes. For example, as shown illustrated in FIG. 1, an accelerometer enclosed by the IMD housing may produce a first axis signal responsive to acceleration along vector 2, which may be aligned with the longitudinal axis of IMD 14. A second axis signal may be responsive to acceleration along vector 1, aligned with a first radial axis of IMD 14. A third axis signal may be responsive to acceleration along vector 3, which may be aligned with a second radial axis of IMD 14. Vectors 1, 2 and 3 may be orthogonal vectors though a multi-dimensional accelerometer included in IMD 14 may sense acceleration along multiple axes that are not necessarily arranged orthogonally in other examples. Furthermore, in some examples, an accelerometer included in IMD 14 may be a one dimensional or two dimensional accelerometer or have more than three axis signals corresponding to more than three vector directions.

The acceleration signals produced by the accelerometer along one, two or all three vectors 1, 2 and 3 may be used by processing circuitry of IMD 14 in detecting dislodgement of IMD 14. If a fixation member anchoring IMD 14 at its implant site becomes wholly or partially dislodged or weakens or fractures, the motion of IMD 14 may increase due to forces imposed on IMD 14 by moving blood, cardiac and/or other moving anatomical structures, gravity and patient physical body motion. Acceleration signals produced by an accelerometer in IMD 14 may change in peak amplitude and/or acceleration signal peaks may become more variable or change in time relative to events of the cardiac cycle. The frequency content of the acceleration signals may change as mobility of IMD 14 changes with altered holding forces of the fixation member(s) of IMD 14. As described below, IMD 14 may process and analyze acceleration signals produced by an accelerometer to detect changes in amplitude, frequency and/or other variations in the accelerometer signal that may be indicative of dislodgement of IMD 14.

In some examples, IMD system 10 may include a second device 12 that may be a second IMD but could be an external device in some examples. The second device 12 may be fixedly implanted or positioned in a location relative to the patient's anatomy such that, with respect to the patient's anatomy, the orientation of the second device 12 can be expected to be stable over time. The second device 12 may be implanted subcutaneously, submuscularly or within the thorax, abdomen or other location but may be implanted in a location where it is subjected to less motion than IMD 14, less likely to become dislodged, or less likely to change orientation relative to the patient's anatomy. The second device 12 may be implanted so that it is not in contact with the heart 8. The second device 12 may be embodied as a variety of medical devices such as a pacemaker, defibrillator, cardiac monitor, electroencephalogram (EEG) monitor, glucose monitor, drug pump, neurostimulator, etc. The second device 12 includes at least a single-axis accelerometer or a multi-axis accelerometer for sensing acceleration signals. The second device 12 may be configured to determine a device orientation signal from one or more accelerometer axis signals. In the conceptual diagram of FIG. 1, second device 12 is configured for sensing acceleration in three dimensions corresponding to vectors 1, 2 and 3. The vectors 1, 2 and 3 may or may not be aligned relative to the vectors 1, 2, and 3, shown conceptually, of the accelerometer included in IMD 14.

As further described below, IMD 14 may be configured to receive orientation signals from second device 12. As such, IMD 14 and second device 12 may be configured to communicate directly with each other, as indicated by communication link 26. IMD 14 and second device 12 may communicate via radio frequency (RF) telemetry or tissue conduction communication (TCC). In other examples, IMD 14 and second device 12 may communicate indirectly. For instance, second device 12 may transmit orientation signals to an external device, e.g., external device 20. External device 20 may transmit the orientation signals to IMD 14. In still other examples, external device 20 may receive orientation signals from both IMD 14 and second device 12 for analysis for detecting dislodgement of IMD 14 according to the techniques disclosed herein. For instance, processing circuitry included in medical device system 10 may be configured to determine a difference between orientation signals determined from the three axis signals of the accelerometers included in IMD 14 and the second medical device 12. The difference(s) between IMD orientation signals and the orientation signals from the second device 12 at a baseline time point may change compared to differences between the IMD orientation signals and the orientation signals from the second device 12 at a later time due to dislodgement of IMD 14. A change in the difference between an IMD orientation signal and second device orientation signal may be used in detecting dislodgement of IMD 14 in some examples, as further described below.

IMD 14 may be capable of bidirectional wireless communication with external device 20 for programming sensing and pacing control parameters. External device 20 is often referred to as a “programmer” because it is typically used by a physician, technician, nurse, clinician or other qualified user for programming operating parameters in an IMD. Operating parameters, including sensing and therapy delivery control parameters, may be programmed into IMD 14 by a user interacting with external device 20. External device 20 may be located in a clinic, hospital or other medical facility. External device 20 may alternatively be embodied as a home monitor or a handheld device that may be used in a medical facility, in the patient's home, or another location.

External device 20 may include a processor 52, memory 53, display unit 54, user interface 56 and telemetry unit 58. Processor 52 controls external device operations and processes data and signals received from IMD 14. Display unit 54 may generate a display, which may include a graphical user interface, of data and information relating to IMD functions to a user for reviewing IMD operation and programmed parameters. Display unit 54 may generate a display that includes cardiac signals and/or data derived therefrom, cardiac pacing timing markers, cardiac pacing history and/or other physiological data, patient data or device-related data that may be stored by IMD 14 and transmitted to external device 20 during an interrogation session. For example, IMD 14 may generate an output for transmission by a communication circuit of IMD 14 to external device 20 including pacing and sensing event histories, device operating parameters and device diagnostic data.

External device 20 may receive a dislodgement alert transmitted from IMD 14 when dislodgement is detected according to the techniques disclosed herein. An accelerometer signal episode recorded at the time of dislodgement detection and/or data derived from the accelerometer signal for detecting the dislodgement may be transmitted from IMD 14 in conjunction with the alert. Any of the various metrics described herein that can be used by IMD 14 for detecting dislodgement may be stored in memory of IMD 14 over time for transmission to external device 20. A history or log of determined metrics can provide a clinician or other expert an overview of how the metrics changed over time leading up to the time of dislodgement detection. Dislodgement related signals and/or data acquired by IMD 14 (and in some examples second device 12) may be displayed to a user by display unit 54. Other patient related data and/or device related data, such as a history of electrode impedance measurements, pacing capture threshold, pacing history, etc. may be transmitted to external device 20 upon receipt of an interrogation command.

User interface 56 may include a mouse, touch screen, keypad or the like to enable a user to interact with external device 20 to initiate a telemetry session with IMD 14 for retrieving data from and/or transmitting data to the IMD 14, including programmable parameters for controlling sensing and pacing functions. In some examples, various parameters used by IMD 14 in detecting dislodgement, such as dislodgement thresholds, dislodgement monitoring schedule, or other parameters, may be programmable by a user interacting with user interface 56. Telemetry unit 58 includes a transceiver and antenna configured for bidirectional communication with a telemetry circuit included in IMD 14 and is configured to operate in conjunction with processor 52 for sending and receiving data relating to IMD functions via communication link 24. Telemetry unit 58 may also be referred to as a “communication circuit” and may function according to any communication protocol implemented to receive and send communication signals from and to IMD 14 and second device 12, when present.

Telemetry unit 58 may establish a wireless bidirectional communication link 24 with IMD 14. Communication link 24 may be established using an RF link such as BLUETOOTH®, Wi-Fi, Medical Implant Communication Service (MICS) or other communication bandwidth. In some examples, external device 20 may include a programming head that is placed proximate IMD 14 to establish and maintain a communication link 24, and in other examples external device 20 and IMD 14 may be configured to communicate using a distance telemetry algorithm and circuitry that does not require the use of a programming head and does not require user intervention to maintain a communication link.

It is contemplated that external device 20 may be in wired or wireless connection to a communications network via a telemetry circuit that includes a transceiver and antenna or via a hardwired communication line for transferring data to a centralized database or computer to allow remote management of the patient. Remote patient management systems including a centralized patient database may enable a clinician to view data relating to sensing and pacing functions performed by IMD 14. For example, a dislodgement alert received by external device 20 may be transmitted to a centralized database or physician or hospital network or computer for notifying a healthcare provider that further evaluation or corrective action may be required.

While IMD 14 is described herein as a leadless pacemaker, it is to be understood that the dislodgement detection methods disclosed herein may be implemented in a variety of IMDs which may include cardiac monitors, neurological monitors, implantable sensors, pacemakers, neurological or muscle stimulators, drug pumps, or the like. The IMD may sense physiological signals other than a motion signal. The IMD may or may not be capable of delivering a therapy. IMD 14 is shown as a leadless device however the methods for detecting dislodgement may be implemented in conjunction with a device having one or more medical leads extending from the IMD.

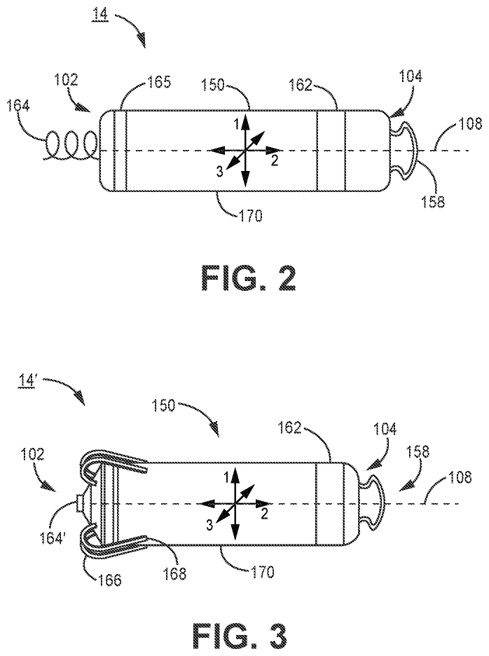

FIG. 2 is a conceptual diagram of the IMD 14 shown in FIG. 1 according to one example. IMD 14 includes a housing 150 having a distal end 102 and a proximal end 104. The lateral sidewall 170 of housing 150 extending from distal end 102 to proximal end 104 may have a generally circular cross-section to facilitate transvenous delivery, e.g., via a catheter. Distal end 102 is referred to as “distal” in that it is expected to be the leading end as IMD 14 is advanced through a delivery tool, such as a catheter, and placed against a targeted pacing site. In other examples, housing 150 may have a generally prismatic shape. The housing 150 encloses the electronics and a power supply for sensing cardiac signals, producing pacing pulses and controlling therapy delivery and other functions of IMD 14 as described herein.

IMD 14 is shown including electrodes 162, 164 and 165 spaced apart along the housing 150 of IMD 14 for sensing cardiac electrical signals and delivering pacing pulses. In various examples, IMD 14 may include more than three or less than three electrodes as shown here. Electrode 164 is shown as a tip electrode extending from distal end 102 of housing 150. Electrodes 162 and 165 are shown as ring electrodes along the lateral sidewall 170 of housing 150. Electrodes 162 and 165 may be ring electrodes circumscribing the lateral sidewall 170, for example adjacent proximal end 104 and adjacent distal end 102, respectively.

Tip electrode 164 is shown as a screw-in helical electrode which serves as the IMD fixation member, providing fixation of IMD 14 at an implant site as well as serving as a pacing and/or sensing electrode. Electrode 164 may be advanced from within the right atrial chamber to a ventricular pacing site, e.g., for delivering pacing to the His-Purkinje conduction system and/or for pacing of ventricular septal myocardial tissue. Electrode 164 may be advanced into patient tissue at any desired implant site by rotation of IMD 14 using an implant and delivery tool. The distal tip of electrode 164 may pierce the tissue at the implant site and the helical electrode 164 may be advanced to a desired tissue depth as IMD 14 is rotated, e.g., by rotating a delivery tool coupled to IMD 14.

Tip electrode 164 may serve as a cathode electrode with ring electrode 162 serving as a return anode for delivering ventricular pacing pulses. Tip electrode 164 and ring electrode 162 may be used as a bipolar pair for ventricular pacing and for receiving a ventricular electrical signal from which R-waves attendant to ventricular depolarizations can be sensed by sensing circuitry enclosed by housing 150. Ring electrodes 162 and 165 may form a second anode and cathode pair for bipolar atrial pacing and/or sensing an atrial electrical signal from which P-waves attendant to atrial depolarizations can be sensed by the sensing circuitry enclosed by housing 150. Electrodes 162, 164 and 165 may be, without limitation, titanium, platinum, iridium or alloys thereof and may include a low polarizing coating, such as titanium nitride, iridium oxide, ruthenium oxide, platinum black, among others.

Electrodes 162, 164 and 165 may be positioned at locations along IMD 14 other than the locations shown. Examples of various pacing electrode arrangements for providing cardiac pacing along the native conduction system of the heart and/or ventricular myocardium are generally disclosed in U.S. Pat. No. 11,426,578 (Yang, et al.) and U.S. Pat. No. 11,007,369 (Sheldon, et al.), both of which are incorporated herein by reference in their entirety.

Housing 150 is formed from a biocompatible material, such as a stainless steel or titanium alloy. In some examples, the housing 150 may include an insulating coating. Examples of insulating coatings include parylene, urethane, PEEK, or polyimide, among others. The entirety of the housing 150 may be insulated, but only electrodes 162, 164 and 165 uninsulated. Tip electrode 164, serving as a cathode electrode, can be coupled to internal circuitry, e.g., a pacing pulse generator and cardiac electrical signal sensing circuitry enclosed by housing 150, via an electrical feedthrough crossing housing 150. Electrodes 162 and 165 may be formed as a conductive portion of housing 150 defining respective ring electrodes that are electrically isolated from each other and from the other portions of the housing 150 as generally shown in FIG. 2.

Housing 150 encloses pacemaker electronics and a power source, which provides power to the pacemaker electronics. As described below, the enclosed electronics can include various circuits and components for sensing cardiac signals, generating pacing pulses and controlling signal sensing, therapy delivery and other functions of IMD 14 as described below in conjunction with FIG. 3. A motion sensor may be implemented as an accelerometer enclosed within housing 150 in some examples. The accelerometer may provide a sensed acceleration signal to a processor included in IMD 14 for signal processing and analysis for detecting IMD dislodgement. In some examples, the acceleration signals may be used for other cardiac monitoring and pacing control functions. For example, cardiac mechanical event signals corresponding to atrial systole, ventricular systole, and/or ventricular diastole may be sensed from acceleration signals sensed by an accelerometer included in IMD 14. When IMD 14 is provided for ventricular pacing, for example, IMD 14 may be implanted in the RA or the right ventricle and sense atrial systolic events from the accelerometer signal for use in controlling the timing of ventricular pacing pulses in an atrial synchronous pacing mode. In other examples, the accelerometer signal may be analyzed for determining a patient physical activity metric for controlling rate response pacing by increasing a pacing rate during periods of increased physical activity.

The accelerometer may be a three-dimensional accelerometer. In some examples, the accelerometer may have one “longitudinal” axis that is parallel to or aligned with the longitudinal axis 108 of IMD 14 for sensing acceleration in the direction of vector 2 shown conceptually in FIG. 2. The accelerometer may have two axes orthogonal to the longitudinal axis 108 that extend in radial directions relative to the longitudinal axis 108, e.g., for sensing acceleration in the direction of vectors 1 and 3 shown conceptually in FIG. 2. Practice of the techniques disclosed herein, however, are not limited to a particular orientation of the accelerometer within or along housing 150. In other examples, a one-dimensional accelerometer may be used to sense a motion signal from which acceleration signals can be processed and analyzed according to techniques disclosed herein for detecting dislodgement. In still other examples, a two dimensional accelerometer or other multi-dimensional accelerometer configured to produce more than 3 axis signals may be used. Each axis of the single or multi-dimensional accelerometer may be defined by a piezoelectric element, micro-electrical mechanical system (MEMS) device or other sensor element capable of producing an electrical signal in response to changes in acceleration imparted on the sensor element, e.g., by converting the acceleration to a force or displacement that is converted to the electrical signal. In a multi-dimensional accelerometer, the sensor elements may be arranged orthogonally with each sensor element axis orthogonal relative to the other sensor element axes. Orthogonal arrangement of the elements of a multi-axis accelerometer, however, is not necessarily required.

Each sensor element may produce an acceleration axis signal corresponding to a vector aligned with the axis of the sensor element, e.g., vectors 1, 2 and 3 as shown in FIG. 2. IMD 14 may be configured to select an axis signal of a multi-axis accelerometer (also referred to as a “multi-dimensional” accelerometer) for use in sensing an IMD orientation signal with respect to gravity and/or an acceleration signal corresponding to motion of IMD 14. In some cases one, two or all three axis signals produced by a three dimensional accelerometer may be combined to determine a signal corresponding to motion and/or the orientation of IMD 14 in two or three dimensions for use in detecting dislodgement of IMD 14 according to methods disclosed herein. It is to be understood that one, two or all three axis signals may be processed and analyzed for other IMD functions, e.g., for detecting atrial systolic events for controlling atrial synchronized ventricular pacing delivered by IMD 14 and/or confirming sensed P-waves and/or for determining a patient physical activity metric for controlling the pacing rate during a rate response pacing mode.

IMD 14 may include features for facilitating deployment to and fixation at an implant site. For example, IMD 14 may optionally include a delivery tool interface 158. Delivery tool interface 158 may be located at the proximal end 104 of IMD 14 and is configured to connect to a delivery device, such as a catheter, used to position IMD 14 at an implant location during an implantation procedure. The delivery tool interface may enable a clinician to advance, retract and steer IMD 14 to an implant site and rotate IMD 14 to advance the helical tip electrode 164 into the cardiac tissue. Helical tip electrode 164 in this example provides fixation of IMD 14 at the implant site. In other examples, however, IMD 14 may include a set of fixation tines or other fixation members to secure IMD 14 to cardiac tissue.

FIG. 3 is a conceptual diagram of an IMD having a fixation member provided as one or more fixation tines. IMD 14′ shown in FIG. 3 may be implanted in or on an atrial or ventricular heart chamber as generally described above. In this case, instead of an active fixation electrode, like helical electrode 164, IMD 14′ incudes a tip electrode 164′ that can serve as a pacing and/or sensing cathode electrode as described above but does not serve as a fixation member for anchoring IMD 14′ at the implant site. Instead, IMD 14′ is provided with one or more fixation tines 166.

Fixation tines 166 may be deployed from the distal end of a catheter positioned at a desired implantation site for IMD 14′. Fixation tines 166 may have a normally curved position as shown in FIG. 3. The fixation tines 166, however, can be deployable from a spring-loaded position in which fixation tines 166 are held in a generally straightened position within a delivery tool during advancement to an implant site. The distal tips 168 of the fixation tines 166 can point distally, away from distal end 102, when fixation tines 166 are held in a straightened position within the confines of a delivery catheter or other delivery tool. The distal tips 168 of fixation tines 166 may penetrate the tissue at the implant site as IMD 14′ is pushed out of the distal end of the delivery catheter. The fixation tines 166 may curve back toward the proximal end 104 of IMD 14′ from the spring-loaded straightened position as IMD 14′ is advanced further out and released from the delivery catheter. The fixation tines 166 advanced into the tissue at the implant site capture tissue within the curved portions of the fixation tines 166 as the distal tips 168 of the fixation tines 166 bend or curve backward, in the general direction of proximal end 104. Fixation tines 166 assume their normal curved or bent position, as shown in FIG. 3, thereby actively engaging tissue at the implant site to securely anchor IMD 14′. Fixation tines 166 can provide a deployment energy sufficient to penetrate a desired patient tissue and secure an IMD, e.g., IMD 14′, to the patient tissue. In some examples, the fixation tines 166 and other fixation members described herein allow for removal from a patient tissue followed by redeployment, e.g., to adjust the position of the IMD relative to the patient tissue. Fixation tines 166 may correspond to examples of deployable fixation tines generally disclosed in U.S. Pat. No. 10,112,045 (Anderson, et al.), incorporated herein by reference in its entirety.

It is to be understood that the length, shape, mechanism of fixation and other attributes of an IMD fixation member (or members) may be adapted for a particular implant location and IMD configuration. Various fixation members that may be used to anchor an IMD at an implant site can include, but are not limited to, tines, hooks, helices, or other members that may penetrate and/or engage with tissue, actively or passively, at the implant site. The dislodgement detection methods disclosed herein are not limited to practice with a particular type of fixation member.

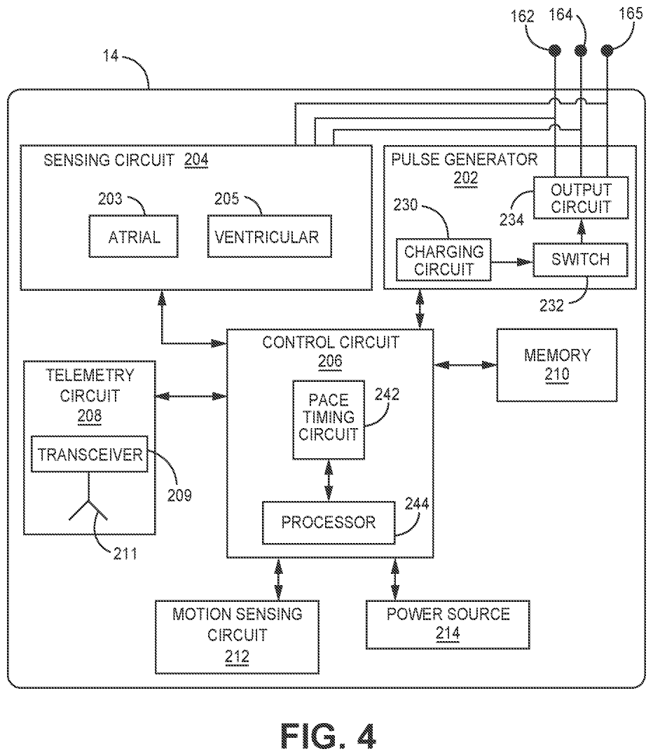

FIG. 4 is a conceptual diagram of an example configuration of IMD 14 according to some examples. IMD 14 may include a pulse generator 202, a cardiac electrical signal sensing circuit 204, a control circuit 206, telemetry circuit 208, memory 210, motion sensing circuit 212 and a power source 214. The various circuits represented in FIG. 4 may be combined on one or more integrated circuit boards which include an application specific integrated circuit (ASIC), an electronic circuit, a processor (shared, dedicated, or group) and memory that execute one or more software or firmware programs, a combinational logic circuit, state machine or other suitable components that provide the described functionality.

Cardiac electrical signal sensing circuit 204, referred to hereafter as “sensing circuit” 204, may be configured to receive at least one cardiac electrical signal via electrodes coupled to IMD 14, e.g., via tip electrode 164 and ring electrode 162. When ring electrode 165 is present, a cardiac electrical signal may be received via electrodes 162 and 165 and/or electrodes 164 and 165. As such, sensing circuit 204 may have multiple sensing channels, e.g., an atrial sensing channel 203 and a ventricular sensing channel 205. Sensing circuit 204 may include switching circuitry for coupling a sensing electrode pair to a respective sensing channel 203 or 205. Sensing channels 203 and 205 may include filters, amplifiers, analog-to-digital converters (ADCs), rectifiers, sense amplifiers, comparators, and/or other circuitry for sensing cardiac event signals, e.g., P-waves and/or R-waves, and producing sensed cardiac event signals, e.g., atrial sensed event signals (Asense signals) and ventricular sensed event signals (Vsense signals), that are passed to control circuit 206. Sensing circuit 204 may be configured to pass a filtered and amplified multi-bit digital cardiac electrogram (EGM) signal to control circuit 206, e.g., from one or both of atrial and ventricular sensing channels 203 and 205. The EGM signal(s) may be processed and analyzed by control circuit 206 for determining a heart rhythm and/or stored in memory 210 as cardiac signal episodes that can be transmitted by telemetry circuit 208, e.g., to external device 20 (shown in FIG. 1).

Control circuit 206 may include a pace timing circuit 242 and processor 244. Control circuit 206 may receive Vsense signals and Asense signals from sensing circuit 204 for use in controlling the timing of cardiac pacing pulses. Vsense signals may be passed from sensing circuit 204 to control circuit 206 in response to ventricular sensing channel 205 sensing a ventricular event signal to indicate the timing of a suspected R-wave. Asense signals may be passed from sensing circuit 204 to control circuit 206 in response to atrial sensing channel 203 sensing an atrial event signal to indicate the timing of a suspected P-wave.

Each channel of sensing circuit 204 may include a peak track and hold circuit or other peak detection circuitry for determining the peak amplitude of sensed R-waves and/or sensed P-waves. In some examples, the amplitudes of cardiac event signals, e.g., R-wave and/or P-waves, sensed by sensing circuit 204 may be used by control circuit 206 in detecting dislodgement in combination with processing and analysis of an acceleration signal received from motion sensing circuit 212.

Processor 244 may pass sensing control parameters to sensing circuit 204 for use in sensing cardiac event signals from the cardiac electrical signal(s). For example, one or more blanking periods, refractory periods, atrial sensitivity, ventricular sensitivity, and other control parameters used by sensing circuit 204 for applying a sensing threshold amplitude for sensing cardiac event signals attendant to the depolarizations of the myocardium may be passed to sensing circuit 204 from processor 244.

Processor 244 may include one or more clocks for generating clock signals that are used by pace timing circuit 242 to time out various pacing intervals for providing ventricular pacing according to an operating pacing mode. Depending on the operating pacing mode of control circuit 206, pace timing circuit 242 may start a pacing interval to schedule a pacing pulse. Control circuit 206 may be configured to operate in a variety of programmable and/or automatically switchable pacing modes. During an atrial synchronous ventricular pacing mode, which may be denoted as a DDD or VDD pacing mode for example, ventricular pacing pulses may be delivered synchronously with atrial pacing pulses and/or received Asense signals. For example, in response to receiving an Asense signal, pace timing circuit 242 may start an AV pacing interval to control the timing of an atrial synchronous ventricular pacing pulse. When a ventricular pacing pulse is delivered by pulse generator 202 upon expiration of the AV pacing interval, pace timing circuit 242 may start a ventricular pacing interval to schedule a ventricular pacing pulse. During atrial synchronous ventricular pacing, if an Asense signal is not received prior to the expiration of a ventricular pacing interval, pulse generator 202 may deliver an asynchronous ventricular pacing pulse and restart the ventricular pacing interval. The scheduled ventricular pacing pulse can be inhibited if an Asense signal is received (or an atrial pacing pulse is delivered) before the ventricular pacing interval expires. The pending pacing pulse may be cancelled and an atrial synchronous triggered ventricular pacing pulse can be delivered at the AV pacing interval from the Asense signal (or delivered atrial pacing pulse).

In response to receiving a Vsense signal from sensing circuit 204, pace timing circuit 242 may inhibit a pending ventricular pacing pulse scheduled at the ventricular pacing interval (or scheduled at an AV pacing interval) and restart the ventricular pacing interval. The ventricular pacing interval may be a lower rate interval (LRI) corresponding to a programmed minimum or base ventricular pacing rate. In other instances, the ventricular pacing interval may be a temporary ventricular pacing interval set to a rate smoothing interval to avoid an abrupt change in ventricular rate. In other instances, the ventricular pacing interval may be a temporary rate response pacing interval set to provide rate response pacing during increased patient physical activity (which may be detected by control circuit 206 based on an acceleration signal received from motion sensing circuit 212).

Pulse generator 202 generates electrical pacing pulses that can be delivered to pace the ventricles of the patient's heart via cathode electrode 164 and return anode electrode 162. In examples including atrial pacing capabilities, pulse generator 202 may generate electrical pacing pulses for pacing the atria, e.g., using electrodes 165 and 162. In other examples, IMD 14 may be a single chamber atrial or ventricular pacemaker and deliver pacing pulses, atrial or ventricular, via the tip electrode 164 and a proximal ring electrode 165 or 162. In addition to providing control signals to pace timing circuit 242 and pulse generator 202 for controlling the timing of pacing pulses, processor 244 may retrieve programmable pacing control parameters from memory 210, such as a pacing mode, pacing pulse amplitude and pacing pulse width, which can be used in controlling pulse generator 202 in generating pacing pulses according to a pacing mode.

Pulse generator 202 may include charging circuit 230, switching circuit 232 and an output circuit 234. Charging circuit 230 is configured to receive current from power source 214 and may include a holding capacitor that may be charged to a pacing pulse amplitude, e.g., under the control of a voltage regulator included in charging circuit 230. The pacing pulse amplitude may be set based on a control signal from control circuit 206. Switching circuit 232 may control when the holding capacitor of charging circuit 230 is coupled to the output circuit 234 for delivering the pacing pulse. For example, switching circuit 232 may include a switch that is activated by a timing signal received from pace timing circuit 242 upon expiration of a pacing escape interval and kept closed for a programmed pacing pulse width to enable discharging of the holding capacitor of charging circuit 230. The holding capacitor, previously charged to the pacing pulse voltage amplitude, can be discharged across electrodes 164 and 162 (or 165 and 162) through an output capacitor of output circuit 234 for the programmed pacing pulse duration.

It is to be understood that when IMD 14 is configured for dual chamber pacing, pulse generator 202 may be configured for delivering both atrial and ventricular pacing pulses under the control of pace timing circuit 242. The atrial pacing pulses are generated by pulse generator 202 according to an atrial pacing pulse amplitude and pulse width. The ventricular pacing pulses are generated by pulse generator 202 according to a ventricular pacing pulse amplitude and pulse width. Pulse generator 202 may include an atrial pacing channel and a ventricular pacing channel that may be controlled separately to deliver atrial pacing pulses upon expiration of atrial pacing intervals and ventricular pacing pulses upon expiration of AV pacing intervals and/or ventricular pacing intervals, respectively. The separate atrial pacing channel and ventricular pacing channel may include some shared circuitry for generating and delivering pacing pulses. For example, atrial and ventricular pacing channels may include shared output circuitry that is selectively coupled to the appropriate pacing electrode pair via switching circuitry included in output circuit 234.

Control circuit 206 may confirm that pacing capture occurs when a pacing evoked response can be detected from cardiac electrical signals sensed by sensing circuit 204 and/or cardiac mechanical signals sensed from an acceleration signal received from motion sensing circuit 212. When an evoked response is not detected, control circuit 206 may detect loss of capture, e.g., no pacing evoked depolarization of the myocardium in response to a delivered pacing pulse. Control circuit 206 may be configured to perform a pacing capture threshold test by controlling pulse generator 202 to deliver pacing pulses at one or more pacing pulse outputs (e.g., one or more pacing pulse amplitudes and/or pulse widths). The lowest pacing pulse output at which pacing capture is detected can be determined to be the pacing capture threshold.

Control circuit 206 may be configured to control pulse generator and sensing circuit 204 to perform an electrode impedance measurement by delivering an excitation signal via drive signal pair of electrodes and measuring the resulting signal via a recording pair of electrodes. In other examples, control circuit 206 may determine a pacing electrode impedance by determining the voltage drop from the leading edge of a delivered pacing pulse to the trailing edge of the delivered pacing pulse.

A change in electrode impedance, a change in pacing capture threshold, and/or a loss of capture may be an electrical indicator of IMD dislodgement. Any of these electrical indicators of IMD dislodgement may be used by control circuit 206 in combination with acceleration signal metrics described below for detecting IMD dislodgement by control circuit 206.

IMD 14 may include a motion sensing circuit 212 for sensing motion imparted on IMD 14 due to cardiac motion, blood flow, patient physical activity, and/or changes in patient posture relative to gravity. Motion sensing circuit 212 may include a single-axis or multi-axis accelerometer as described above for producing acceleration signals along one or more axes. Motion sensing circuit 212 may include one or more filters, amplifiers, analog to digital converters, rectifiers and/or other components for passing an acceleration signal corresponding to each accelerometer axis individually or in combinations of two axes or all three axes to control circuit 206.

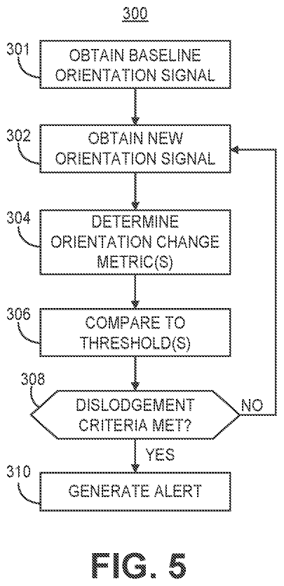

Acceleration signals received from motion sensor 212 may be processed and analyzed by control circuit 206 for detecting dislodgement of IMD 14 according to the techniques disclosed herein. The orientation of IMD 14 relative to gravity, relative to a baseline orientation, or relative to another implanted device, e.g., second device 12 shown in FIG. 1, may be determined by control circuit 206 based on the acceleration signals. Accelerometer signals may be low pass filtered to provide an averaged signal component along one or more axes of the accelerometer with respect to gravity to obtain an IMD orientation signal. As described below, e.g., in conjunction with FIG. 5, a change in the orientation signal relative to a baseline orientation signal and/or relative to a second device orientation signal may be determined by control circuit 206 and used in detecting IMD dislodgement.

Additionally or alternatively, control circuit 206 may receive acceleration signals from one or more axes of the accelerometer for determining changes in acceleration amplitude. A combination, e.g., a summation, a weighted summation, a difference, a product, a ratio, or other combination, of the rectified or non-rectified axis signals corresponding to vectors 1, 2 and 3 may be determined by control circuit 206. These acceleration signals may be analyzed by control circuit 206 for detecting changes in the peak amplitude of the acceleration signals, the timing of the acceleration signal peaks relative to cardiac events, and/or changes in the frequency content of the acceleration signals for detecting IMD dislodgement. Various methods for processing and analyzing the acceleration signals received from motion sensing circuit 212 for detecting IMD dislodgement are described below in conjunction with the accompanying flow charts and diagrams.

The accelerometer signal received from motion sensing circuit 212 may be used for purposes other than detecting IMD dislodgement in some examples. For instance, control circuit 206 may receive a rectified acceleration signal from motion sensing circuit 212 and determine a patient physical activity metric from the acceleration signal, e.g., by summing acceleration signal sample point amplitudes over an activity metric time interval. The activity metric may be converted to a target heart rate to meet the patient's metabolic demand. The target heart rate may be converted to a sensor indicated rate (SIR) based on an SIR transfer function that includes a lower rate set point and an activities of daily living (ADL) range and a maximum upper rate, for example. During a rate response pacing mode, pulse generator 202 may be controlled by control circuit 206 to deliver atrial or ventricular pacing pulses at a rate response pacing rate determined based on the SIR.

Memory 210 may include computer-readable instructions that, when executed by control circuit 206, cause control circuit 206 to perform various functions attributed throughout this disclosure to IMD 14. The computer-readable instructions may be encoded within memory 210. Memory 210 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal.

Memory 210 may store acceleration signal metrics determined by control circuit 206 so that control circuit 206 can track trends in the acceleration signal metrics for use in detecting IMD dislodgement. In some examples, memory 210 includes a buffer that stores acceleration signal metrics determined by control circuit 206 for use by control circuit 206 in determining a long term trend value of the metric, e.g., a long term average, median, maximum, minimum, range or other representative value. The long term representative values of acceleration metrics may be used by control circuit 206 for setting thresholds that can be applied to new acceleration metrics as they are determined for detecting IMD dislodgement. Memory 210 may store one or more episodes of acceleration signals. A history of acceleration signals and/or acceleration signal metrics may be stored in memory 210 for transmission via telemetry circuit 208.

Telemetry circuit 208 includes a transceiver 209 and antenna 211 for transferring and receiving data via an RF communication link. Telemetry circuit 208 may be capable of bi-directional communication with external device 20 (FIG. 1) as described above and, at least in some examples, a second device 12 as shown in FIG. 1. A dislodgement detection alert signal, acceleration signals and/or data derived therefrom may be transmitted by telemetry circuit 208 to external device 20. Programmable control parameters and algorithms for detecting IMD dislodgement, sensing acceleration signals, sensing cardiac event signals and controlling pacing therapies delivered by pulse generator 202 may be received by telemetry circuit 208 and stored in memory 210 for access by control circuit 206. Telemetry circuit 208 may be referred to as a “communication circuit” because it can be configured to communicate wirelessly, e.g., via RF communication, TCC or other communication methods, for transmitting communication signals to and/or receiving communication signals from external device 20 and/or second device 12. In some examples, IMD 14 may communicate with second device 12 via TCC and communicate with external device 20 via RF communication, for example.

Power source 214 provides power to each of the other circuits and components of IMD 14 as required. Power source 214 may include one or more energy storage devices, such as one or more rechargeable or non-rechargeable batteries. The connections between power source 214 and other IMD circuits and components are not shown in FIG. 4 for the sake of clarity but are to be understood from the general block diagram of FIG. 4. Power source 214 may provide power as needed to pulse generator 202, sensing circuit 204, telemetry circuit 208, memory 210 and motion sensing circuit 212.

The functions attributed to IMD 14 herein may be embodied as one or more processors, controllers, hardware, firmware, software, or any combination thereof. Depiction of different features as specific circuitry is intended to highlight different functional aspects and does not necessarily imply that such functions must be realized by separate hardware, firmware or software components or by any particular circuit architecture. Rather, functionality associated with one or more circuits described herein may be performed by separate hardware, firmware or software components, or integrated within common hardware, firmware or software components. For example, a process of detecting IMD dislodgement may be implemented in control circuit 206 executing instructions stored in memory 210 and relying on input from motion sensing circuit 212 and in some examples from sensing circuit 204. In some examples, processing circuitry may be included in motion sensing circuit 212 and control circuit 206 for cooperatively determining acceleration signal metrics and detecting IMD dislodgement according to the techniques disclosed herein. Providing software, hardware, and/or firmware to accomplish the described functionality in the context of any modern medical device, given the disclosure herein, is within the abilities of one of skill in the art.

FIG. 5 is a flow chart 300 of a method for detecting IMD dislodgement according to some examples. At block 301, control circuit 206 may obtain a baseline orientation signal. The baseline orientation signal may be obtained at the time of initial device implant. The baseline orientation signal, however, may be obtained or updated at any time after the implant procedure, e.g., during a follow up visit or upon a command received from an external device, e.g., external device 20 shown in FIG. 1. Motion sensing circuit 212 (shown in FIG. 4) may include a low pass filter, e.g., a 0.5 to 1 Hz low pass filter, to provide the averaged acceleration signal component with respect to gravity from each accelerometer axis or at least one selected accelerometer axis. In some examples, control circuit 206 determines the average amplitude of the low pass filtered acceleration signal from each accelerometer axis signal as the baseline orientation signal, which may be stored in memory 210. The low pass filtered acceleration signal may be averaged over a time period of one cardiac cycle, a specified number of cardiac cycles, 0.5 seconds, 1 second, 2 seconds, 5 seconds, 10 seconds or other desired time interval, which may extend over multiple cardiac cycles, to obtain an orientation signal average representative of the prevailing position of IMD 14 along each of one or more accelerometer axes relative to gravity.

At block 302, control circuit 206 may obtain a new orientation signal from the accelerometer signal during a different, subsequent time period, according to a dislodgement monitoring protocol. The posture of the patient at the time of obtaining the baseline orientation signal may be accounted for and replicated for obtaining subsequent new orientation signals at block 302. For example, the baseline orientation signal may be obtained at the time of implant, after IMD 14 has been released and detached from the delivery tool system. This can provide a baseline orientation signal in a known posture because the IMD is generally implanted when the patient is in a supine position. Subsequent new orientation signals may be obtained at block 302 at a scheduled time of day when the patient is expected to be in the same posture, e.g., a supine position. For example, a new orientation signal may be obtained one or more times during nighttime hours when the patient is expected to be asleep.

In another example, a baseline orientation signal may be obtained as the median or average of each acceleration axis signal sensed over successive time periods during nighttime hours. To illustrate, the low pass filtered signal from each accelerometer axis may be obtained over successive time periods, e.g., 0.5 to 10 second time periods, at 30 minute intervals between 1am and 4am. The median or average of the average amplitudes of the successive time periods may be determined for each axis signal as the baseline orientation signal at block 301. The baseline orientation signal obtained in this manner may account for variations in orientation due to changes in patient sleeping posture and/or differences between patients in sleeping posture vs. the supine implant position.

In still other examples, the baseline orientation signal may be obtained when the patient is in an upright posture, e.g., a seated upright posture, e.g., when IMD 14 is communicating with external device 20 (shown in FIG. 1). A seated upright or standing posture may be the most common patient posture at the time of transmissions to an external device or a remote patient monitoring network, e.g., via external device 20 shown in FIG. 1. Subsequent new orientation signals may be obtained at block 302 when the IMD 14 has established a communication link with an external device because the patient posture is likely to be the same posture as the baseline posture. The known patient posture condition at the time of obtaining the baseline orientation signal may be stored in IMD memory 210, e.g., as entered from a user interface of external device 20 from a selection of options such as “supine,” “seated,” “left side,” “right side,” etc.

At block 302, control circuit 206 obtains a new orientation signal according to a monitoring protocol. For example, control circuit 206 may obtain a new orientation signal once per hour, once per day, once per week, at one or more scheduled times of day or other scheduled basis. When the baseline orientation signal is obtained by sampling multiple time points, e.g., by averaging the signal obtained over multiple successive time periods during specified nighttime hours, the new orientation signal may be obtained in the same manner.

In some examples, a new orientation signal may be obtained at block 302 in response to a triggering event such as a change in electrode impedance, a change in pacing capture threshold, detection of loss of pacing capture, low amplitude R-waves or P-waves (e.g., less than a threshold difference from a programmed sensitivity) or another indication of suspected dislodgement.

In other instances, a new orientation signal may be obtained at block 302 in response to an on-demand check of the IMD orientation in response to a command entered by a clinician during an in-office follow-up or via communication through the remote patient database network and external device 20. In this case, the patient could be instructed to assume a specified posture, e.g., supine, seated, etc., to match the baseline patient posture condition. A new orientation signal may be obtained after the patient confirms they have assumed the instructed posture or after a countdown or specified time delay.

At block 304, control circuit 206 may determine an orientation change metric from the baseline orientation signal and the new orientation signal. The orientation change metric may be determined as the difference between the average amplitude of each accelerometer axis signal obtained at block 302 and the respective accelerometer axis signal baseline average amplitude obtained at block 301. An orientation signal determined as the average amplitude of each low pass filtered accelerometer axis signal may be referred to as {a(1), a(2), a(3)}, e.g., where a(1), a(2) and a(3) are the average amplitudes corresponding to the axis along respective direction of vectors 1, 2 and 3 as shown in FIG. 2. The average amplitude of an axis signal may be determined over a specified time period that is expected to extend over multiple cardiac cycles. Analogously, the baseline orientation signal determined as the average amplitude of each accelerometer axis signal determined over a specified time period may be referred to as {b(1), b(2), b(3)}. The orientation change metric may be determined at block 304 as the average amplitude differences, e.g., {a(1)-b(1), a(2)-b(2), a(3)-b(3)}. The orientation change metric may be stored in memory 210.

Additionally or alternatively, control circuit 206 may determine an orientation change metric at block 304 based on a combination of differences between the individual average amplitudes determined from each axis signal of the new orientation signal and the baseline orientation signal. For example, control circuit 206 may determine a distance between the point {a(1), a(2), a(3)} and the point {b(1), b(2), b(3)}. The distance may be determined as a “Manhattan” distance D by computing D=|a(1)−b(1)|+|a(2)−b(2)|+|a(3)−b(3)|, though other distance formulas may be used. A difference determined between the amplitudes of the new orientation signal and the baseline orientation signal may generally be referred to as an “orientation amplitude metric.” The orientation amplitude metric may be determined from one or more axis signals of the accelerometer.

Additionally or alternatively, an orientation change metric may be determined as an orientation angle metric at block 304 based on the obtained baseline orientation signal and the new orientation signal. For instance, an angle relative to a selected accelerometer axis 1, 2 or 3 to the vector defined by the point {a(1), a(2), a(3)} may be determined. To illustrate, in three dimensions, the angle θ may be defined as the angle between {a(1), a(2), a(3)} and axis 2 of the accelerometer in an example. The angle Φ may be defined as the angle between the projection of the vector defined by {a(1), a(2), a(3)} in the radial plane defined by the radial axes 1 and 3 and either one of the radial axes 1 and 3. Other conventions may be chosen for defining one or more angles between the vector defined by {a(1), a(2) and a(3)} relative to the accelerometer axes. Control circuit 206 may determine the orientation change metric at block 304 by determining the angle θ and/or the angle Φ for the new orientation signal, determining the angle θ and/or the angle Φ for the baseline orientation signal, and determining the orientation metric as the difference between the new angle θ and the baseline angle θ and/or the difference between the new angle Φ and the baseline angle Φ. An orientation change metric determined as the difference between an angle determined from the baseline orientation signal and an angle determined from a new orientation signal may generally be referred to as an “orientation angle metric.”

In some examples, control circuit 206 may determine an orientation metric as a non-quantitative metric representative of the sign (e.g., positive or negative) of an average amplitude of an axis signal. In some examples, when dislodgement occurs, the sign of the average amplitude of the low pass filtered accelerometer signal from a given axis may change compared to the sign of the baseline average amplitude of the respective accelerometer axis signal. For example, when the fixation member is a rotatable member, such as helical fixation member like the helical electrode 164 shown in FIG. 2, rotation of IMD 14 in a direction that reduces the depth of penetration of fixation member in the tissue can cause an orientation signal sign to change. Control circuit 206 may determine the sign of the average amplitude of the low pass filtered accelerometer signal for one or more of the accelerometer axes at baseline and store the baseline sign in memory 210 (e.g., a “0” for positive and a “1” for negative). Control circuit 206 may determine the sign of the average amplitude of the new orientation signal for each respective axis. Control circuit 206 may determine the difference between the baseline sign and the new sign at block 304 as an orientation change metric. A difference of 0 indicates that no sign change has occurred between the baseline orientation signal and the new orientation signal. An absolute difference of 1 indicates a change between the baseline orientation signal and the new orientation signal for a given accelerometer axis signal and may be an indication of IMD dislodgement, e.g., due to rotation of IMD 14. An orientation change metric determined from the signs of the average amplitudes of the low pass filtered accelerometer signals can be referred to as an “orientation sign metric.” The orientation sign metric may be determined for one or more accelerometer axis signals. In some examples, the sum of the absolute differences between the baseline sign and the new sign for two or all three accelerometer axes may be determined. For instance, if the sign of all three axis signals changes from the baseline orientation signal to the new orientation signal, the sign change metric can be 3.

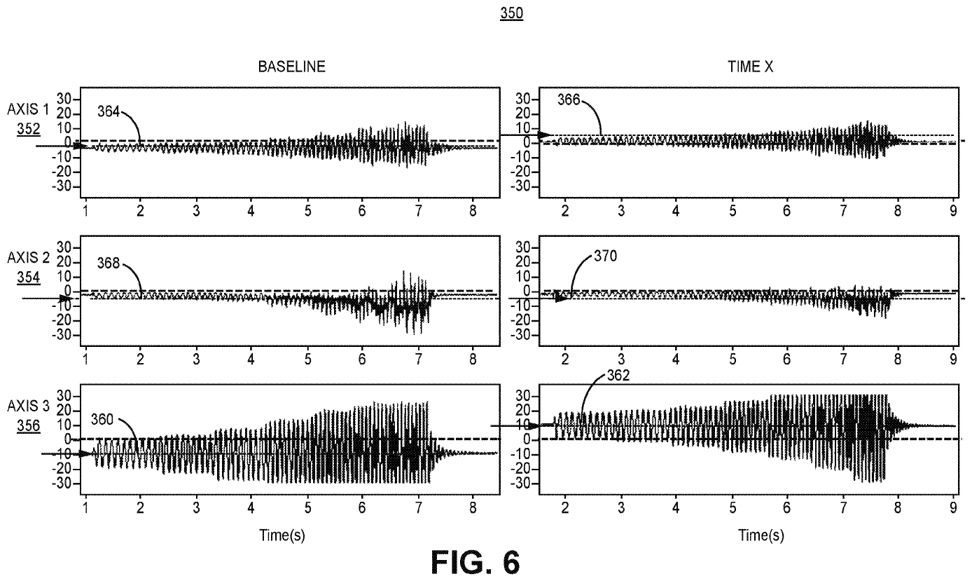

FIG. 6 is a diagram 350 of accelerometer axis signals obtained at baseline and at a later time point X. The axis signals 352, 354 and 356 may correspond to respective vectors 1, 2 and 3 as generally illustrated in FIG. 2. The axis signals 352, 354 and 356 may correspond to signals obtained during bench testing of the accelerometer when it is powered up and positioned in two different orientations relative to gravity at a baseline time point and the later time point X. The average amplitude of acceleration signal 356 for axis 3 is observed to undergo a sign change. The average amplitude 360 of the accelerometer axis signal 356 at baseline has a negative sign. The average amplitude 362 of the accelerometer axis signal 356 at time X has a positive sign. This change in sign can indicate a rotation of the IMD. If the longitudinal axis 2 is not the axis that is predominately aligned with the direction of gravity, one or both of the average amplitudes of the low pass filtered radial axis signals 1 and 3 (352 and 356) may change in sign when IMD 14 rotates relative to a starting baseline position.

As observed in FIG. 6, the absolute value of the average amplitude 364 at baseline and the average amplitude 366 at time X for axis 1 signal 352 is relatively smaller than the average amplitude of the accelerometer axis 3 signal 356. The sign of the signal 352 for axis 1, however, is negative at baseline and changes to positive at time X. Accordingly, the change in signs for axis 1 and axis 3 and no sign change for axis 2 354 (as shown by negative average amplitudes 368 and 370 at both baseline and at time X) can be used by control circuit 206 in detecting IMD dislodgement due to rotation of the IMD compared to its baseline position in some examples. Whether or not an orientation sign metric is determined as an orientation change metric and, if so, the axis signals for which an orientation sign metric is/are determined may be selectively programmed in an IMD based on the type of fixation member of the IMD and/or the relative position of the IMD with respect to gravity at baseline.

Referring again to FIG. 5, as described above, control circuit 206 may determine one or more orientation change metrics at block 304, which may include one or more of an orientation amplitude metric, an orientation angle metric and/or an orientation sign metric according to any of the examples described above. At block 306, control circuit 206 may compare the orientation change metrics to respective dislodgement thresholds.

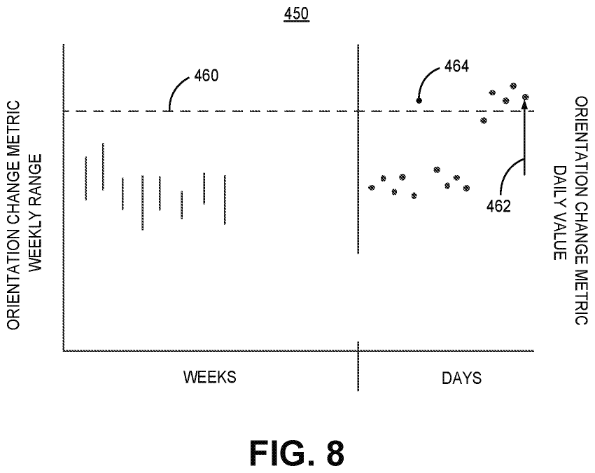

The threshold applied to a given orientation change metric may be programmed and stored in memory 210, user programmable, and/or may be determined by control circuit 206 based on orientation signals obtained at baseline and/or one or more later time points. For example, an orientation change metric threshold may be determined by control circuit 206 as a percentage or offset from an orientation change metric determined from the baseline orientation signal. In some cases, an orientation change metric threshold can be determined based on a long term average, median, maximum, minimum, range or other representative value of a long term trend of given orientation change metric determined at successive time periods over a long term trending internal, e.g., a daily, weekly, or monthly trending interval. Thus, a daily, weekly or monthly average, median, maximum and/or minimum of the orientation change metric may be determined by control circuit 206 from orientation change metrics stored in memory 210. Control circuit 206 may establish a threshold that can be applied to a new orientation change metric where the threshold is based on the daily, weekly, monthly (or other long term trending time interval) average, median, maximum, minimum, or other representative value. Examples of determining a dislodgement detection threshold based on a long term trend value are described below in conjunction with FIG. 8.

Control circuit 206 may determine if the orientation change metrics meet dislodgement criteria at block 308. In order to avoid a false positive dislodgement detection, control circuit 80 may determine when an orientation metric meets a dislodgement threshold at least a minimum number of times that successive new orientation signals are obtained. For example, orientation change metrics may be determined hourly. Control circuit 206 may determine that dislodgement criteria are met at block 308 when at least 4, 8, 12 or other specified number of the hourly orientation change metrics meet the dislodgement threshold. In another example, orientation change metrics may be determined daily. Control circuit 206 may determine that dislodgement criteria are met at block 308 when at least 2, 3, 4, 5 or other specified number of the daily orientation change metrics meet the dislodgement threshold. The specified number of times that new orientation change metrics are required to meet dislodgement detection thresholds may or may not be required to be consecutively determined orientation change metrics. In an illustrative example, three consecutive daily orientation change metrics may be determined to meet dislodgement criteria at block 308. In another illustrative example, three out of the most recent five daily orientation change metrics may be determined to meet dislodgement criteria at block 308. When the orientation change metric meets a dislodgement threshold a single time, control circuit 206 may not determine that dislodgement criteria are met at block 308. A single orientation change metric meeting a dislodgement threshold may be a false positive indication of IMD dislodgement. To avoid a false positive detection of IMD dislodgement, control circuit 206 may detect IMD dislodgement when the orientation change metric determined from the axis signals for each of multiple time periods meets a threshold at block 306 for at least two or more of the multiple time periods.

If dislodgement criteria are not met at block 308, control circuit 206 may return to block 302 to obtain the next new orientation signal. If dislodgement criteria are met at block 308, control circuit 206 may detect IMD dislodgement and generate an alert at block 310. The alert may be transmitted by telemetry circuit 208. Orientation change metrics and/or orientation signals corresponding to the baseline and/or one or more new orientation signals that led to the IMD dislodgement detection being made may be transmitted by telemetry circuit 208.

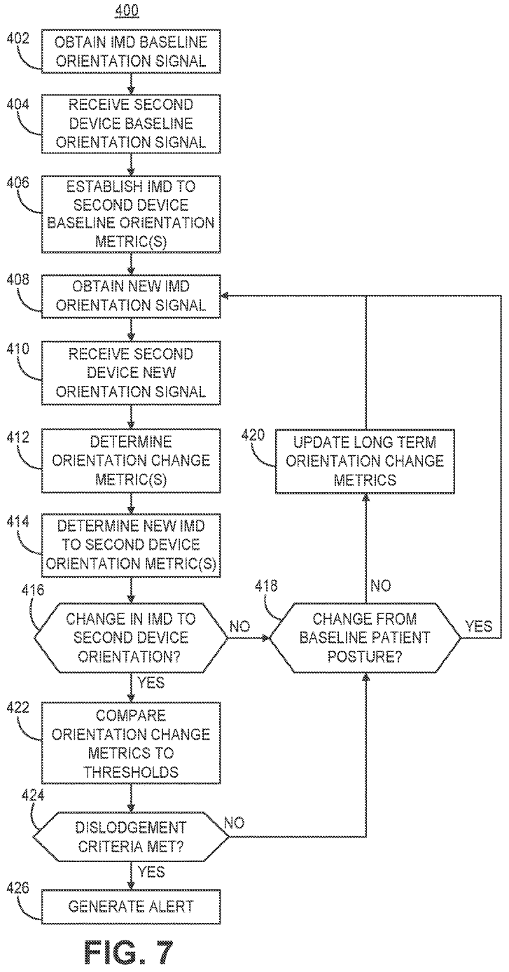

FIG. 7 is a flow chart 400 of a method for detecting IMD dislodgement based on orientation change metrics according to another example. At block 402, IMD control circuit 206 may obtain a baseline orientation signal as described above in conjunction with FIG. 5. At block 404, control circuit 80 may receive a baseline orientation signal from a second device, e.g., second device 12 shown in FIG. 1, which may be co-implanted with IMD 14 but at another location than IMD 14. When IMD 14 is implanted in or on the patient's heart, for example, the second device may be implanted away from the patient's heart, e.g., subcutaneously or submuscularly outside the thorax. The baseline orientation signal received from the second device may be the averaged amplitude of one or more axis signals of the accelerometer of the second device that are low pass filtered for obtaining the average signal component relative to gravity. The second device may be configured to obtain a baseline orientation signal in the same manner as the IMD though the second device may obtain the baseline orientation signal in a different manner, e.g., different filtering, different sampling rate, different averaging time interval, or other differences. The second device baseline orientation signal is obtained during the same patient posture as the IMD baseline orientation signal, however, to promote valid comparisons of new orientation signals obtained at later time points for a given patient posture.

The second device baseline orientation signal may be received by IMD 14 in response to an orientation signal request transmitted to the second device from IMD 14. In other examples, the second device baseline orientation signal may be received by IMD 14 from external device 20 (FIG. 1), which may have requested and received the second device baseline orientation signal from the second device. The second device baseline orientation signal may be received in the form of the average amplitude of the low pass filtered axis signal of one or more axis signals of the second device accelerometer. For example the second device baseline orientation signal may be represented by {d(1), d(2), d(3)}, where d(1), d(2) and d(3) are the average amplitudes of respective low pass filtered axis signals corresponding to axis 1, axis 2 and axis 3 of the second device accelerometer sensed over an averaging time period (which may extend over multiple cardiac cycles and may be equal to a specified time period that IMD 14 determines an average amplitude of an axis signal for obtaining an orientation signal). IMD control circuit 206 may receive the values of d(1), d(2) and/or d(3) from the second device, indirectly or directly.

When the baseline orientation signal for the second device is established at a time when the IMD baseline orientation signal is obtained, or at least during the same patient posture, the relationship between the second device orientation and the IMD orientation associated with the given patient posture can be determined and stored by IMD 14. While the IMD orientation signal can change with changes in patient posture and the second device orientation signal can change with changes in patient posture, a relationship between the orientation signals of IMD 14 and the second device is not expected to change unless the IMD 14 becomes dislodged and changes position relative to the second device. It is assumed that the second device and IMD 14 are subjected to the same or similar orientation change relative to gravity with changes in patient posture. For example, second device 12 may be implanted along the upper torso of the patient when IMD 14 is implanted in or on the patient's heart.

At block 406, after receiving the second device baseline orientation signal control circuit 206 may establish a baseline orientation metric representative of the relationship between the IMD baseline orientation signal and the second device baseline orientation signal. The baseline orientation metric may be the difference between the average amplitude of the low pass filtered signals of the IMD accelerometer and the second device accelerometer for one or more accelerometer axis signals. For the sake of illustration, the second device may have a three axis accelerometer. If the baseline orientation signal for IMD 14 is {b(1), b(2), b(3)} and the baseline orientation signal of the second device is {d(1), d(2), d(3)}, the baseline orientation metric between IMD 14 and the second device may be determined by control circuit 206 as {(b1-d1), (b2-d2), (b3-d3)}, as an example. In other examples, the difference between the amplitudes of a single axis or the difference between the amplitudes of two axes may be determined.

Control circuit 206 may determine one or more baseline orientation metrics at block 404 which may include an amplitude difference metric, an angle difference metric, and/or a sign difference metric in various examples. An amplitude difference metric can be determined as a difference between the amplitudes of one or more axis signals of the IMD accelerometer and the second device accelerometer, e.g., as given above. An amplitude difference metric could additionally or alternatively be determined as the distance between the three dimensional points defined by {b(1), b(2), b(3)} and {d(1), d(2), d(3)}, e.g., the “Manhattan” distance. An angle difference metric may be determined by determining an angle between the vectors defined by the points {b(1), b(2), b(3)} and {d(1), d(2), d(3)}. An angle difference metric may be determined as a difference between the angles θ and/or the angles Φ determined for each of the vectors defined by the respective three-dimensional points {b(1), b(2), b(3)} and {d(1), d(2), d(3)}. A sign difference metric may be determined by comparing the signs of b(1) and d(1), b(2) and d(2), and/or b(3) and d(3). Any of the example orientation change metrics described above in conjunction with FIG. 5 for determining a representative value of the relationship between a new IMD orientation signal and the IMD baseline orientation signal could be determined as a baseline orientation metric representative of the relationship between the IMD baseline orientation signal and the second device baseline orientation signal. The baseline orientation metrics determined by control circuit 206 at block 406 can be stored in memory 210 for use in detecting IMD dislodgement.

At block 408, control circuit 206 obtains a new IMD orientation signal. The new IMD orientation signal may be obtained at a scheduled time, in response to a triggering event or upon a command from external device 20. At block 410, control circuit 206 may receive a second device new orientation signal. Control circuit 206 may control telemetry circuit 208 to transmit an orientation signal request to the second device and receive the orientation signal transmitted by the second device in response to the request. In other examples, the new IMD orientation signal may be obtained at block 406 during a communication session with external device 20. External device 20 may transmit a request to the second device for an orientation signal, receive the new orientation signal from the second device and transmit the second device new orientation signal to IMD 14. In still other examples, the second device may be programmed to obtain a new orientation signal according to the same dislodgement monitoring protocol as IMD 14 such that a second device new orientation signal is available when a new IMD orientation signal is being obtained. The second device may transmit the second device new orientation signal to IMD 14.

At block 412, control circuit 206 may determine orientation change metrics based on the new IMD orientation signal and the IMD baseline orientation signal. Any of the orientation change metrics described above in conjunction with FIG. 5 may be determined at block 414 based on the IMD baseline orientation signal (obtained at block 402) and the new IMD orientation signal (obtained at block 408). At block 414, control circuit 206 may determine a new IMD to second device orientation metric representative of the new IMD orientation signal relative to the second device new orientation signal. Any of the example orientation metrics determined as baseline orientation metrics at block 406 described above may be determined as new orientation metrics at block 414 based on the new IMD and second device new orientation signals.

At block 416, control circuit 206 may compare the IMD to second device orientation metrics to the IMD to second device baseline orientation metrics established at block 406. If the IMD to second device orientation metric(s) match the IMD to second device baseline orientation metric(s) (e.g., within a specified threshold range or percentage of each other), control circuit 206 may determine that IMD dislodgement is unlikely. No further evaluation of the orientation change metrics may be required. Control circuit 206 may advance to block 418 in response to the new IMD to second device orientation metric(s) being relatively unchanged compared to the baseline orientation metric(s).

At block 418, control circuit 206 may determine if the second device new orientation signal has changed relative to the second device baseline orientation signal. A change in the second device new orientation signal relative to the second device baseline orientation signal can indicate that the patient's posture when the second device new orientation signal was obtained is different than the patient's posture when the second device baseline orientation signal was obtained. If the patient's posture is different, the new IMD orientation signal may not be relevant to detecting IMD dislodgement due a different patient posture compared to the baseline patient posture. The new IMD orientation signal may not be used for updating a long term trend value in the IMD orientation metrics. When control circuit 206 detects a change from the baseline patient posture based on a comparison of the second device new orientation signal to the second device baseline signal (“yes” branch of block 418), control circuit 206 may return to block 408 from block 418 to obtain the next new IMD orientation signal at a next subsequent time period according to the dislodgement monitoring protocol. Control circuit 206 does not detect IMD dislodgement and may not update a long term trend of the orientation change metrics when a change from the baseline patient posture is detected at block 418.

When the second device new orientation signal matches the second device baseline orientation signal (e.g., within a threshold range or percentage), control circuit 206 does not detect a change from the baseline patient posture at block 418. Control circuit 206 may determine that the patient posture associated with the new IMD orientation signal and the second device new orientation signal corresponds to the baseline patient posture. In this case, the new IMD orientation signal can be relevant to updating long term trends of orientation change metrics. Control circuit 206 may update a long term trend value of one or more orientation change metrics at block 420 using the orientation change metrics determined at block 412. However, control circuit 206 may not evaluate or detect IMD dislodgement based on the orientation change metrics when the new IMD to second device orientation metrics have not changed compared to the baseline IMD to second device orientation metrics (“no” branch of block 416).

Referring again to block 416, when control circuit 206 determines a change in the IMD to second device orientation metric(s) compared to the baseline orientation metrics, control circuit 206 may advance to block 422. A change in the IMD to second device orientation metric(s) relative to the baseline IMD to second device orientation metric(s) determined at block 406 may be detected when one or more of the new IMD to second device orientation metrics is different from the respective baseline IMD to second device orientation metric by at least a threshold difference or percentage. A change in the orientation of IMD 14 relative to the second device may be an indication of dislodgement of IMD 14. As such, at block 422, control circuit 422 may compare the orientation change metrics determined at block 412 based on the new IMD orientation signal and the IMD baseline orientation signal.

Control circuit 206 may determine that dislodgement criteria are met at block 424 based on a comparison of the orientation change metrics to respective thresholds according to any of the examples provided above in conjunction with FIG. 5. For instance, when a change in IMD to second device orientation is detected (“yes” branch of block 416) and an orientation amplitude metric, an orientation angle metric and/or an orientation sign metric determined from the IMD baseline orientation signal and new IMD orientation signal meet respective thresholds for each of at least a threshold number of subsequent time periods, control circuit 206 may detect IMD dislodgement at block 424. When dislodgement criteria are met, control circuit 206 may generate a dislodgement alert at block 426.

In the example shown in FIG. 7, when dislodgement criteria are not met (“no” branch of block 424), control circuit 206 may advance to block 418. If a comparison of the second device new orientation signal to the second device baseline orientation signal indicates that the patient posture associated with the new orientation signals matches the baseline patient posture (“no” branch of block 418), control circuit 206 may optionally use the new orientation change metrics (between the IMD baseline orientation signal and new IMD orientation signal) to update long term trend values of the orientation change metrics at block 420. As described above, the long term trend values of the orientation change metrics may be used in determining a threshold that is applied to new orientation change metrics. If a comparison of the second device new orientation signal to the second device baseline orientation signal indicates that the patient posture associated with the new orientation signals does not match the baseline patient posture (“yes” branch of block 418), control circuit 206 may return to block 408 without updating the long term trend values of the orientation change metrics.

In other examples, when a change between the IMD to second device orientation is detected at block 416 (“yes” branch), control circuit 206 may not use the new orientation change metrics for updating long term trends in the orientation change metrics. When dislodgement criteria are unmet, control circuit 206 may return to block 408 to obtain the next new IMD orientation signal at the next successive time period according to the dislodgement monitoring protocol without checking for a posture change at block 418 and without updating the long term trends in the orientation change metrics at block 420.

FIG. 8 is a diagram 450 of a plot of long term trends of an orientation change metric (left side of graph) and new, recent orientation change metrics determined at scheduled monitoring intervals (right side of graph). In this example, an orientation change metric may be determined on a daily basis. The daily orientation change metrics are shown on the right side of the graph for the most recent 15 days. On the left side of the graph, each vertical bar represents a weekly range between a maximum value and minimum value of the daily orientation change metrics determined over a given week. Control circuit 206 may be configured to determine a dislodgement threshold 460 based on the long term trend values, in this case the weekly ranges, of the orientation change metric. Dislodgement threshold 460 may be determined as a percentage or offset greater than the average or median of N weekly maximums, the average or median of N weekly averages, or other representative value of the long term trend of the orientation metrics. In other examples, dislodgement threshold 460 may be determined based on a specified number of daily orientation metrics or all daily orientation metrics determined since a baseline orientation metric or first orientation metric after the baseline is obtained. It is to be understood that the dislodgement threshold may be set to a default value upon IMD implantation until enough data points are obtained for establishing dislodgement threshold 460. As daily orientation metrics are obtained, or after a specified number of daily orientation metrics are obtained, the initial default dislodgement threshold may be adjusted by control circuit 206 to a threshold 460 that is tailored to the patient.

It is to be further understood that in some cases, new orientation change metrics that are determined to be outliers can be rejected for use in updating the long term trend values and threshold 460. For example, each weekly or other long term trend value may be determined as a trimmed mean or trimmed median where the greatest and/or smallest values of the orientation change metric are discarded. In other examples, an orientation change metric may be discarded for use in updating long term trend values when the correct patient posture is not verified, e.g., based on an IMD to second device orientation metric. In some examples, a new orientation change metric is not used to update the long term trend value and threshold 460 when the new orientation change metric exceeds threshold 460.

The dislodgement threshold 460 is shown as a high threshold having a value greater than the weekly ranges of the orientation change metric and is met when a new orientation metric exceeds the dislodgement threshold 460. It is to be understood that depending on the particular metric, the dislodgement threshold may be defined as a low threshold that is less than long term trend values and can be met when an orientation metric is less than the threshold. In still other examples, the dislodgement threshold may be a range defined by a high threshold and a low threshold that may be met when a new orientation metric falls outside the threshold range.

In some examples, multiple (e.g., daily) values of an orientation change metric may be required to meet the dislodgement threshold 460 before control circuit 206 detects dislodgement and performs a response to the dislodgement detection. For example, a single daily value 464 that is greater than threshold 460 may be a false positive and may not result in a dislodgement detection. However, when four out of five daily values meet the threshold 460, control circuit 206 may detect dislodgement as indicated by arrow 462. In this way, a false positive detection of IMD dislodgement is avoided by requiring a persistent change in the orientation change metric determined at the monitoring intervals relative to a threshold based on long term trend values of the orientation change metric (or relative to a default threshold during the early days/weeks after IMD implant as the long term trend values of the orientation change metric are being updated). While daily orientation change metrics are depicted in FIG. 8, it is to be understood that the orientation change metric may be determined at multiple time periods, each time period occurring at a dislodgement monitoring time intervals (e.g., hourly, daily, weekly, monthly, etc.) or scheduled time of day. Each of the multiple time periods may extend over multiple cardiac cycles.

Long term trend values and daily values of orientation change metrics are depicted in FIG. 8. However, it is to be understood from this illustrative example that long term trend values of other acceleration signal metrics determined by control circuit 206 for use in detecting IMD dislodgement may be determined and tracked in the same or similar manner as any of the examples described in conjunction with FIG. 8. A dislodgement threshold applied to a new acceleration signal metric determined at a subsequent, later time period according to a dislodgement monitoring protocol can be determined based on the long term trend values of the acceleration signal metric in a manner that is analogous to the examples described here in conjunction with FIG. 8 with regard to an orientation change metric. As such, the methods described in conjunction with FIG. 8 for tracking long term trend values, updating long term trend values based on new acceleration signal metrics, and determining a dislodgement threshold based on the long term trend values can generally be applied to any of the acceleration signal metrics described herein for use in detecting IMD dislodgement.

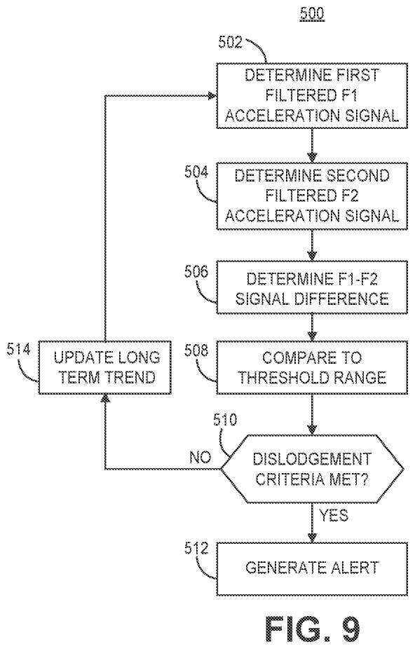

FIG. 9 is a flow chart 500 of a method for detecting IMD dislodgement according to some examples. At block 502, control circuit 206 determines a first filtered acceleration signal (F1 signal). The F1 signal can be determined by filtering the accelerometer axis signals using a low frequency band pass filter or a low pass filter. For example, the accelerometer axis signals may be filtered using a 5 to 30 Hz, 8 to 25 Hz, or 10 to 20 Hz bandpass filter as examples. A low pass filter cut off frequency may be 10 Hz, 15 Hz, 20 Hz, 25 Hz or 30 Hz as examples. The time aligned sample points of each of the low frequency bandpass filtered axis signals (or low pass filtered axis signals) can be rectified and summed to obtain the F1 signal at block 502, which may also be referred to herein as a “first filtered rectified summation signal” or a “first rectified signal.” Other combinations of the rectified axis signals may be determined such as a product of the axis signals, a difference, a weighted summation of the axis signals where each axis signal may have a different weighting factor or other mathematical combination of the axis signals. The F1 signal can be obtained over a specified time period or number of cardiac cycles. For instance the F1 signal may be obtained over 10 to 100 cardiac cycles, 20 to 80 cardiac cycles or 24 to 72 cardiac cycles in various examples. The cardiac cycles may be identified based on a cardiac electrical signal sensed by sensing circuit 204 (e.g., based on sensed R-waves or sensed P-waves) and/or based on pacing pulses delivered by pulse generator 202. Alternatively, the F1 signal may be obtained over a time period of 10 seconds, 20 seconds, 30 seconds, one minute or other specified time period expected to include multiple cardiac cycles. The F1 signal may be filtered to include relatively low frequency acceleration signals associated with motion of IMD 14, in contrast to the orientation signals that represent the DC component of the axis signals with respect to gravity.

At block 504, control circuit 206 may determine a second filtered acceleration signal (F2 signal). The F2 signal can be determined by filtering the accelerometer axis signals using a relatively higher frequency bandpass filter than the low frequency bandpass filter used to obtain the F1 signal. For example, the accelerometer axis signals may be filtered using a 20 to 50 Hz, 25 to 40 Hz, or 25 to 35 Hz bandpass filter as examples. In one example, the F1 signal is obtained by applying a 10 to 20 Hz bandpass filter to the three accelerometer axis signals. The F2 signal is obtained by applying a 25-35 Hz bandpass filter to the three accelerometer axis signals. In other examples, the F2 signal could be determined by filtering the accelerometer axis signals using a high pass filter, e.g., having a high pass filter cut off frequency of 20 Hz, 25 Hz, or 30 Hz as examples. The time aligned sample points of each of the high frequency bandpass filtered axis signals (or high pass filtered axis signals) can be rectified and summed to obtain the F2 signal at block 504, which may also be referred to as a “second filtered rectified summation signal,” though the F2 signal may be determined as other mathematical combinations of the high frequency bandpass or high pass filtered axis signals as described above. The F2 signal may be acquired over the same, simultaneous time period or number of cardiac cycles as the F1 signal so that the F2 signal and the F1 signal can be time aligned. In other examples, the F2 signal and the F1 signal could be acquired over two non-simultaneous time periods, e.g., over sequential sets of a specified number of cardiac cycles, during alternating sets of cardiac cycles or specified time periods, etc.

Control circuit 206 may determine a difference between the F1 and F2 signals at block 506. In some examples, control circuit 206 determines the F1-F2 signal difference by determining a maximum peak amplitude of the F1 signal during each cardiac cycle of multiple cardiac cycles and determining the maximum peak amplitude of the F2 signal during each cardiac cycle of multiple cardiac cycles, e.g., 10 to 100 cycles. The mean, median, or highest of the maximum peak amplitudes determined from the F1 signal maximum peak amplitudes may be determined as the F1peakamp. The mean, median, or highest of the maximum peak amplitudes determined from the F2 signal maximum peak amplitudes may be determined as F2peakamp. The F1-F2 signal difference may be determined at block 506 as the difference between the F1peakamp and the F2peakamp. In other examples, the difference between the F1 maximum peak amplitude and the F2 maximum peak amplitude may be determined for each cardiac cycle on a beat by beat basis. The mean, median, maximum, minimum or other representative value of the individual cycle differences between the F1 maximum peak amplitude and the F2 maximum peak amplitude may be determined as the F1-F2 signal difference at block 506. In still other examples, the difference between the average amplitude of the F1 signal obtained over the specified time period (e.g., spanning multiple cardiac cycles) and the average amplitude of the F2 signal obtained over the specified time period may be determined as the F1-F2 signal difference at block 506.

At block 508, the F1-F2 signal difference may be compared to a dislodgement threshold. The dislodgement threshold may be determined by control circuit 206 based on a long term trend value of the F1-F2 signal differences, e.g., as generally described above in conjunction with FIG. 8. In this case, the F1-F2 signal differences determined as acceleration signal metrics can be representative of a shift in the frequency content of the accelerometer axis signals. The F1-F2 signal difference can be referred to as a “frequency metric” which can be determined on a daily basis or according to another monitoring interval so that weekly ranges (or other long term trending interval values) of the F1-F2 signal differences can be determined. Multiple weekly (or other long term trending interval) medians, maximums, means, trimmed medians, or other representative values of the F1-F2 signal differences determined over long term trending intervals may be determined and used for establishing a threshold that is applied to the new F1-F2 signal differences determined at each successive monitoring interval, e.g., daily.

The F1-F2 signal differences may change in sign over time. As such, when a median weekly F1-F2 signal difference is determined, for example, the median difference may shift positively or negatively due to IMD dislodgement. The threshold applied to the daily F1-F2 signal difference may be a threshold range in this case such that the F1-F2 signal difference may meet the dislodgement threshold if it falls above or below the threshold range.

At block 510, control circuit 206 may determine when dislodgement criteria are met. For example, when a threshold number of multiple successive daily F1-F2 differences meet the dislodgement threshold range (e.g., on four out of 5 days, on three consecutive days, etc.), control circuit 206 may detect dislodgement at block 512 and generate a dislodgement alert. When dislodgement criteria are not met, control circuit 206 may store the new F1-F2 difference for use in updating a long term trend value at block 514. The updated long term trend value may be subsequently used by control circuit 206 to update the dislodgement threshold range. Control circuit 206 may return to block 502 for determining the next F1 signal according to the scheduled (or triggered) monitoring time. The F1-F2 signal difference may be stored in memory 210 but not used for updating the long term trend value when the F1-F2 signal difference meets the dislodgement threshold requirement (but the threshold number of multiple new F1-F2 signal differences has not yet been reached for detecting dislodgement at block 510).

In the example described here, control circuit 206 determines the maximum peak amplitude of the F1 signal and the F2 signal during each cardiac cycle of multiple cardiac cycles, which may be consecutive or non-consecutive cycles. It is contemplated that the F1-F2 signals may be obtained over a single cardiac cycle at scheduled monitoring intervals in some examples. Furthermore, it is contemplated that the F1-F2 difference may be determined as a difference signal over one or more cycles rather than the difference between the maximum peak amplitudes determined from each of multiple cardiac cycles. The difference signal may be determined by subtracting the time-aligned sample points of the F1 signal from the F2 signal (or vice versa) over one or more cardiac cycles. The difference signal may be compared to a threshold range and if the difference signal falls outside the threshold range a specified number of times for each of a specified number of daily (or other monitoring interval) checks, the dislodgement criteria may be met at block 512. Other methods may be employed for determining a difference between the F1 signal and the F2 signal that indicates a shift in the frequency content of the accelerometer axis signals that could indicate IMD dislodgement.

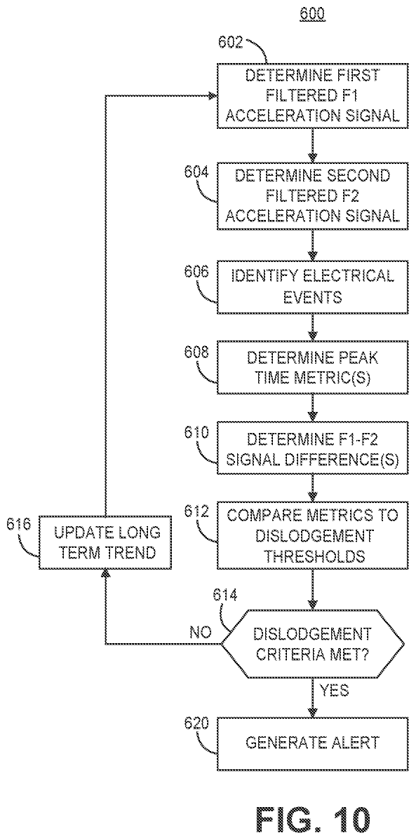

FIG. 10 is a flow chart 600 of another method for detecting IMD dislodgement according to some examples. At block 602, control circuit 206 may determine an F1 signal according to the techniques described above. At block 604, control circuit 206 may determine an F2 signal according to the techniques described above. In other examples, only the F1 signal, only the F2 signal or a different filtered rectified summation signal (summation of the three rectified accelerometer signals or another mathematical combination of the three filtered accelerometer signals) may be determined. For example, instead of the F1 and F2 signals determined as described above, control circuit 206 may filter the accelerometer axis signals using a relatively wider bandpass, e.g., 10 Hz to 50 Hz or 15 Hz to 35 Hz as examples. For the sake of illustration, however, the process of flow chart 600 is described in conjunction with determining the F1 signal and the F2 signal.

The F1 and F2 signals may be obtained over a specified time interval or number of cardiac cycles. At block 606, control circuit 206 may identify cardiac electrical events that occur during the specified time interval or associated with the specified number of cardiac cycles. Control circuit 206 may identify R-waves sensed by sensing circuit 204, P-waves sensed by sensing circuit 204, atrial pacing pulses delivered by pulse generator 202, and/or ventricular pacing pulses delivered by pulse generator 202.

At block 608, control circuit 206 may determine one or more peak time metrics. Control circuit 206 may determine a peak time metric by determining the time from an identified cardiac electrical event to one or more peaks of the F1 signal during a given cardiac cycle and/or from the identified cardiac electrical event to one or more peaks of the F2 signal during the cardiac cycle. The peak time relative to an identified cardiac electrical event (sensed or paced) may be determined for multiple cardiac cycles for each of the F1 and F2 signals. An average or median value of the peak times may be determined as a peak time metric at block 608 for each of the respective F1 and F2 signals. The peak time metric may be determined for one or both of the F1 signal and the F2 signal. The relative timing between a cardiac electrical event and a peak of the F1 signal and/or F2 signal is expected to be relatively stable when IMD 14 is stably fixed at its implant site. As IMD 14 becomes partially dislodged or after complete dislodgement, the relative timing between cardiac electrical events and peaks of the F1 and/or F2 signals are expected to change from a baseline timing relationship and/or may become more variable between cardiac cycles. As such the peak time metrics determined at block 608 may include a median, mean, maximum, minimum, range, standard deviation and/or other statistical measure.

In some examples, control circuit 206 may determine one or more F1-F2 signal differences at block 610. Any of the signal differences described above in conjunction with FIG. 9 may be determined. Additionally or alternatively, control circuit 206 may determine a difference based on the peak time metrics determined at block 608, e.g., a difference between the median F1 peak time and the median F2 peak time.

At block 612, control circuit 206 may compare the peak time metric(s) to a dislodgement threshold. The dislodgement threshold may be based on a baseline peak time metric that can be determined as the first peak time metric determined by control circuit 206 after IMD 14 implant (or any time the baseline is updated or redetermined). In other examples, the long term trend values of the peak time metrics may be determined and used for establishing a threshold value or range as generally described above in regard to other metrics determined from the acceleration signals for detecting dislodgement. For example, daily peak time metrics may be determined and a weekly median peak time may be determined from the daily peak time metrics. A threshold value or range may be determined by control circuit 206 based on N weekly median peak times, e.g., 4, 6, 8, 10 or 12 weekly median peak times.

In some examples, the F1-F2 difference in peak times may be determined and compared to a dislodgement threshold or range. If any other F1-F2 signal differences are determined at block 610 (e.g., according to any of the examples described in conjunction with FIG. 9), the F1-F2 signal difference(s) may be compared to respective dislodgement thresholds (or ranges) at block 612. Control circuit 206 may determine if dislodgement criteria are met at block 614. Control circuit 206 may avoid detecting a false positive dislodgement by requiring at least a threshold number of successive peak time metrics determined at the scheduled dislodgement monitoring intervals meet the dislodgement threshold or range (e.g., so that a single outlier measurement does not cause a false positive detection). Dislodgement criteria may additionally be applied to one or more F1-F2 signal differences at block 614 as generally described in conjunction with FIG. 9.

When dislodgement criteria are not met, control circuit 206 may update long term trend values of the peak time metrics at block 616, which may be used to update a peak time threshold value or range, e.g., according to the methods generally described above in conjunction with FIG. 8. Control circuit 206 may return to block 602 to determine the F1 signal at the next subsequent time period that dislodgement monitoring is scheduled. When dislodgement criteria are met (“yes” branch of block 614), control circuit 206 may detect IMD dislodgement and generate an alert at block 620.

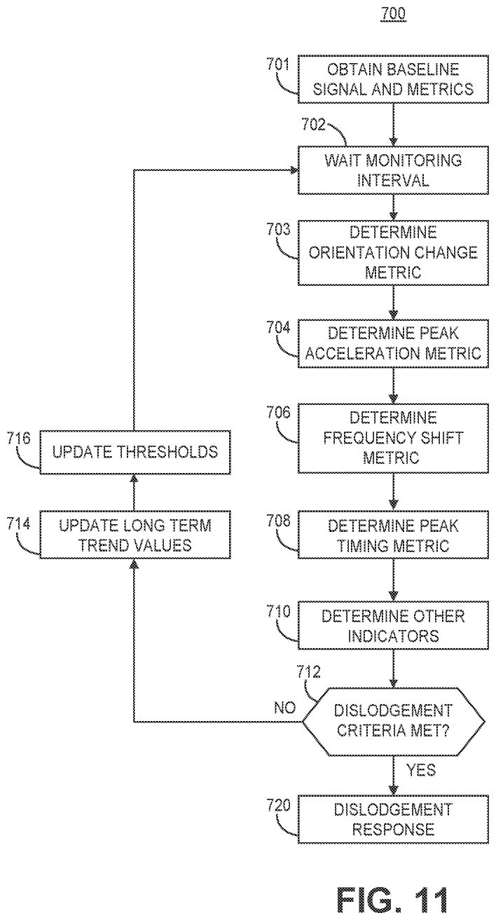

FIG. 11 is a flow chart 700 of a method for detecting IMD dislodgement according to some examples. Control circuit 206 may determine a combination of metrics for detecting dislodgement. The metrics can include any of the example acceleration signal metrics described above (e.g., orientation change metrics, IMD to second device orientation metrics, acceleration signal amplitude metrics, frequency shift metrics, and/or peak timing metrics). In some examples, the combination of metrics can include metrics determined from electrical signals and/or other sensor signals in combination with the acceleration signal metrics determined from the accelerometer signal.

At block 701, control circuit 206 may obtain a baseline orientation signal, a baseline F1 signal, F2 signal or any other baseline filtered acceleration signal. Control circuit 206 may determine baseline acceleration signal metrics at block 701. For example, control circuit 206 may determine a baseline orientation signal as described above in conjunction with FIG. 5. Control circuit 206 may obtain a baseline orientation metric from a received second device orientation signal and the IMD baseline orientation signal as described above in conjunction with FIG. 7. Additionally or alternatively, control circuit 206 may determine a baseline peak acceleration metric determined from a filtered, rectified sum signal. Additionally or alternatively, control circuit 206 may determine a baseline peak timing metric, e.g., from a filtered rectified summation signal, where the acceleration signal peak timing is relative to a cardiac electrical event, e.g., as generally described in conjunction with FIG. 10. Additionally or alternatively, control circuit 206 may determine a baseline F1-F2 difference according to any of the techniques generally described in conjunction with FIGS. 9 and 10.

In some examples, in addition to determining one or more baseline metrics from the accelerometer signal, control circuit 206 may determine a baseline pacing capture threshold, pacing electrode impedance, R-wave amplitude and/or P-wave amplitude. In addition to determining one or more baseline metrics from the accelerometer signal, control circuit 206 may determine a baseline signal and metric from another sensor available in the IMD, such as a gyroscope or magnetometer.

At block 704, control circuit 206 may wait for a monitoring interval to expire. For the sake of illustration, the monitoring interval may be 24 hours so that daily metrics can be determined and stored in memory 210. However, longer or shorter monitoring intervals may be used. Furthermore, it is to be understood that dislodgement monitoring could occur before a monitoring interval has expired if another indicator of possible dislodgement is detected, such as a loss of pacing capture, increased pacing capture threshold, decrease in pacing electrode impedance or a change in another sensor signal. Another indicator of possible dislodgement may trigger processing and analysis of the accelerometer signal for determining if dislodgement criteria are met.

At block 703, control circuit 206 may determine one or more orientation change metrics. Any of the example orientation change metrics described above in conjunction with FIGS. 5, 6 and 7 may be determined at block 703. At block 704, control circuit 206 may determine a peak acceleration metric. The peak acceleration metric may be determined by control circuit 206 by filtering the accelerometer axis signals, e.g., according to any of the example bandpass or cutoff frequencies listed herein, rectifying each of the three axis signals, and combining (e.g., summing) the time aligned sample points of the three axis signals over a specified time interval (e.g., 0.5 seconds to 1 minute) or specified number of cardiac cycles (e.g., one cycle to 100 cycles). The maximum peak amplitude of the filtered, rectified sum of the three axis signals may be determined during each cardiac cycle for example. An average or median of the maximum peak amplitudes may be determined as the peak acceleration metric at block 704. In other examples, a single maximum peak amplitude may be determined from the rectified summation signal, a median or average of the N greatest peak amplitudes may be determined, or a median or average of all peak amplitudes that are greater than a specified threshold amplitude may be determined as the peak acceleration metric at block 704.

At block 706, control circuit 206 may determine a frequency shift metric by determining a difference between an F1 and F2 signal as generally described above in conjunction with FIG. 9. At block 708, control circuit 206 may determine one or more peak timing metrics as generally described above in conjunction with FIG. 10.

In some examples, control circuit 206 may use other indicators of IMD dislodgement for detecting dislodgement in addition to the metrics determined from the accelerometer signal. As such, at block 710, control circuit 206 may determine one or more other indicators of IMD dislodgement. Other indicators of IMD dislodgement may be determined from cardiac electrical signals sensed by sensing circuit 204 or other electrical measurements made using IMD electrodes. Control circuit 206 may determine electrical indicators of IMD dislodgement at block 710, which may include any of a pacing capture threshold, a pacing electrode impedance, an R-wave peak amplitude, a P-wave amplitude and/or evidence of undersensing R-waves or P-waves (e.g., based on increased pacing burden, alternating or variable sensed event intervals (e.g., RR intervals or PP intervals), low or variable R-wave or P-wave amplitudes, or other undersensing evidence). In some examples, IMD 14 may include other sensors, such as a gyroscope or magnetometer, from which a signal metric may be obtained at block 710 as another possible indicator of IMD dislodgement.

At block 712, control circuit 206 may determine if dislodgement criteria are met. Any of the foregoing examples of acceleration signal metrics may be compared to respective dislodgement thresholds or ranges. The dislodgement thresholds or ranges may be programmed by a user, determined by control circuit 206 based on a baseline acceleration signal, and/or determined by control circuit 206 based on the long term trend values of the respective acceleration signal metric. Control circuit 206 may determine that dislodgement criteria are met at block 712 when a combination of acceleration signal metrics meet respective dislodgement thresholds for at least a specified number (e.g., two or other plurality) of multiple successive time periods. Control circuit 206 may determine that the dislodgement criteria are met at block 712 when a combination of acceleration signal metrics meet respective dislodgement thresholds for at least a specified number of multiple successive time periods and at least one other indicator of dislodgement determined at block 710 meets dislodgement criteria. For example, compared to a respective baseline value, an increase in pacing capture threshold, a decrease in pacing electrode impedance (indicating the pacing electrodes may be in the heart chamber blood pool), a decrease in cardiac event signal amplitude (e.g., R-wave amplitude and/or P-wave amplitude), evidence of undersensing (e.g., based on increased pacing burden) and/or loss of capture detection in combination with at least one acceleration signal metric meeting a dislodgement threshold for a specified minimum number of the successive time periods may meet dislodgement criteria applied at block 712.

When dislodgement criteria are not met, the acceleration signal metrics may be used to update long term trend values at block 714. As described above, if an acceleration signal metric meets a dislodgement threshold, it may be stored in memory 210 but not necessarily used for updating the long term trend values at block 714. The updated long term trend values may be used to update dislodgement thresholds at block 716. Control circuit 206 may return to block 702 to wait for the next successive time period for obtaining new acceleration signal metrics according to the dislodgement monitoring protocol.

When control circuit 206 determines that dislodgement criteria are met at block 712, control circuit 206 may perform a dislodgement response at block 720. The dislodgement response may include generating an alert for transmission by telemetry circuit 208. In any of the examples presented herein, when dislodgement criteria are met, control circuit 206 may perform a dislodgement response that includes an adjustment to a control parameter in addition to generating a dislodgement alert. For example, control circuit 206 may adjust a cardiac event sensing control parameter. A sensing electrode vector, a programmed sensitivity, or other control parameter used by sensing circuit 204 for sensing cardiac event signals (which may have a decreased amplitude compared to baseline) may be adjusted.

Additionally or alternatively, control circuit 206 may adjust a cardiac pacing control parameter. Control circuit 206 may adjust a pacing mode, increase a pacing pulse amplitude, increase a pacing pulse width, change a pacing electrode vector, or other adjustment of a control parameter that is used to control the generation of cardiac pacing pulses by pulse generator 202. The pacing mode may be adjusted to a non-sensing pacing mode (fixed pacing rate delivery) when sensing of cardiac event signals may be comprised due to IMD dislodgement. The pacing mode may be adjusted to a non-atrial tracking ventricular pacing mode (e.g., from a VDD pacing mode to a VVI pacing mode) when P-wave sensing may be compromised due to IMD dislodgement. The pacing electrode vector may be changed to a different pacing vector if another pacing electrode vector is available that may be less affected by the IMD dislodgement. The pacing pulse amplitude and/or pacing pulse width may be increased to increase the likelihood of pacing capture and reduce the likelihood of loss of capture due to IMD dislodgement. In other examples, cardiac pacing may be disabled when IMD dislodgement is detected if the patient is not pacemaker dependent because pacing pulse delivery may drain the IMD power source without effectively pacing and capturing the heart due to IMD dislodgement.

Further disclosed herein is the subject matter of the following examples:

Example 1. An IMD including a motion sensor, sensing circuit, pulse generator and control circuit where the motion sensor is configured to sense an axis signal along a plurality of axes of the motion sensor. The sensing circuit is configured to sense cardiac event signals attendant to myocardial depolarizations, and the pulse generator is configured to generate cardiac pacing pulses. The control circuit can be in communication with the motion sensor, the sensing circuit and the pulse generator. The control circuit can be configured to determine a first signal from the axis signals sensed over multiple time periods that each extend over a plurality of cardiac cycles. The control circuit may, for each of the multiple time periods, determine a metric from the first signal and determine that the metrics determined for at least a plurality of the multiple time periods meet a dislodgement threshold. The control circuit may detect dislodgement of the IMD in response to the metrics determined for at least the plurality of the multiple time periods meeting the dislodgement threshold. The IMD may include a communication circuit configured to transmit an IMD dislodgement alert in response to the control circuit detecting the dislodgement.

Example 2. The IMD of example 1 wherein the control circuit is further configured to identify cardiac cycles based on at least one of the cardiac event signals sensed by the sensing circuit or the cardiac pacing pulses generated by the pulse generator. The control circuit may determine, during each of a plurality of the identified cardiac cycles, a peak amplitude of the first signal and determine the metric based on the peak amplitudes.

Example 3. The IMD of example 2 wherein the control circuit is further configured to detect an electrical indicator of dislodgement by determining at least one of: a pacing impedance, a pacing capture threshold, a sensed cardiac event amplitude, a loss of pacing capture, evidence of undersensing of cardiac event signals or an increase in pacing burden. The control circuit may detect the dislodgement of the IMD in response to the metrics determined for at least the plurality of the multiple time periods meeting the dislodgement threshold and detecting the electrical indicator of dislodgement.

Example 4. The IMD of any of examples 2-3 wherein the control circuit is further configured to determine the metric based on the peak amplitudes by, for each of the peak amplitudes, determining a peak time of the peak amplitude relative to the corresponding identified cardiac cycle and determining the metric based on the peak times.

Example 5. The IMD of any of examples 1-4 wherein the control circuit is further configured to filter the axis signals according to a first frequency range and determine the first signal by determining a combination of the axis signals filtered according to the first frequency range. The control circuit may be further configured to filter the axis signals according to a second frequency range and determine a second signal from a combination of the axis signals filtered according to the second frequency range. The control circuit may determine the metric by determining a difference between the first signal and the second signal.

Example 6. The IMD of example 5 wherein the control circuit may be further configured to identify cardiac cycles based on at least one of the cardiac event signals sensed by the sensing circuit or the cardiac pacing pulses generated by the pulse generator. The control circuit may be further configured to determine a first peak amplitude of the first signal during each of a plurality of the identified cardiac cycles and determine a second peak amplitude of the second signal during each of the plurality of the identified cardiac cycles. The control circuit may be further configured to determine the difference between the first signal and the second signal based on the first peak amplitudes and the second peak amplitudes.

Example 7. The IMD of any of examples 1-6 wherein the control circuit is further configured to determine a baseline orientation signal from the axis signals at a baseline time point. The control circuit may be configured to, for at least one of the multiple time periods, determine a new orientation signal from the axis signals. The control circuit may be configured to determine an orientation change metric based on a difference between the new orientation signal and the baseline orientation signal and determine that the orientation change metric meets an orientation change threshold. The control circuit may detect the dislodgement of the IMD in response to the metrics determined for at least the plurality of the multiple time periods meeting the dislodgement threshold and the orientation change metric meeting the orientation change threshold.

Example 8. The IMD of example 7 wherein the control circuit may be further configured to determine the orientation change metric by determining at least one of a difference between an average amplitude of the new orientation signal and an average amplitude of the baseline orientation signal or a difference between an angle defined by an average amplitude of the orientation signal and an angle defined by an average amplitude of the baseline orientation signal.

Example 9. The IMD of example 7 wherein the communication circuit is further configured to receive a second device orientation signal at the baseline time point and at the at least one of the multiple time periods. The control circuit may be further configured to determine a baseline IMD to second device orientation metric from the second device orientation signal received at the baseline time point and the baseline orientation signal. The control circuit may, for the at least one of the multiple time periods, determine a new IMD to second device orientation metric from the second device orientation signal received for the at least one of the multiple time periods and the new orientation signal received for the at least one of the multiple time periods. The control circuit may detect an IMD orientation change based on the baseline IMD to second device orientation metric and the new IMD to second device orientation metric. The control circuit may detect the dislodgement of the IMD in response to the metrics determined for at least the plurality of the multiple time periods meeting the dislodgement threshold and the detected IMD orientation change.

Example 10. The IMD of any of examples 1-9 wherein the control circuit is further configured to detect a sign change in at least one of the axis signals from a baseline time point to at least one of the multiple time periods and detect the dislodgement of the IMD in response to the metrics determined for at least the plurality of the multiple time periods meeting the dislodgement threshold and the detected sign change.

Example 11. The IMD of any of examples 1-10 wherein the control circuit is further configured to determine a long term trend value of the metric and determine the dislodgement threshold based on the long term trend value

Example 12. The IMD of any of examples 1-11 further comprising a housing enclosing the motion sensor, sensing circuit, pulse generator and the control circuit. The IMD further including a plurality of electrodes on the housing and a fixation member extending from the housing for anchoring the IMD at an implant site.

Example 13. The IMD of example 12 wherein at least one electrode of the plurality of electrodes is the fixation member.

Example 14. The IMD of any of examples 1-13, wherein, in response to detecting the dislodgement, the control circuit is configured to adjust at least one of a pacing control parameter used to control the pulse generator in generating the cardiac pacing pulses and/or a sensing control parameter used to control the sensing circuit in sensing the cardiac event signals.

Example 15. The medical device of any of examples 1-14 wherein the control circuit is further configured to determine a rectified axis signal from each of the axis signals sensed along the plurality of axes of the motion sensor and determine the first signal as a combination of the rectified axis signals.

Example 16. A method including sensing an axis signal along a plurality of axes of a motion sensor of an IMD and determining a first signal from the axis signals sensed over multiple time periods. The multiple time periods can each extend over a plurality of cardiac cycles. The method may include determining a metric from the first signal for each of the multiple time periods. The method may include determining that the metrics determined for at least a plurality of the multiple time periods meet a dislodgement threshold and detecting dislodgement of the IMD in response to the metrics determined for at least the plurality of the multiple time periods meeting the dislodgement threshold. The method may include transmitting an IMD dislodgement alert in response to detecting the dislodgement.

Example 17. The method of example 16 may further include identifying cardiac cycles by at least one of sensing cardiac event signals attendant to myocardial depolarizations and/or generating cardiac pacing pulses. The method may include determining a peak amplitude of the first signal during each of a plurality of the identified cardiac cycles and determining the metric based on the peak amplitudes of the first signal.

Example 18. The method of example 17 further including detecting an electrical indicator of dislodgement by determining at least one of: a pacing impedance; a pacing capture threshold; a sensed cardiac event amplitude; a loss of pacing capture; evidence of undersensing of cardiac event signals; or an increase in pacing burden. The method may further include detecting the dislodgement of the IMD in response to the metrics determined for at least the plurality of the multiple time periods meeting the dislodgement threshold and detecting the electrical indicator of dislodgement.

Example 19. The method of any of examples 17-18 further including determining the metric based on the peak amplitudes by for each of the peak amplitudes, determining a peak time of the peak amplitude relative to the corresponding identified cardiac cycle and determining the metric based on the peak times.

Example 20. The method of any of examples 16-19 further including filtering the axis signals according to a first frequency range and determining the first signal by determining a combination of the axis signals filtered according to the first frequency range. The method may further include filtering the axis signals according to a second frequency range and determining a second signal from a combination of the axis signals filtered according to the second frequency range. The method may include determining the metric by determining a difference between the first signal and the second signal.

Example 21. The method of example 20 further comprising identifying cardiac cycles based on at least one of sensing cardiac event signals attendant to myocardial depolarizations and/or generating cardiac pacing pulses. The method may include determining a first peak amplitude of the first signal during each of a plurality of the identified cardiac cycles and, determining a second peak amplitude of the second signal during each of the plurality of the identified cardiac cycles. The method may include determining the difference between the first signal and the second signal based on the first peak amplitudes and the second peak amplitudes.

Example 22. The method of any of examples 16-21 further including determining a baseline orientation signal from the axis signals at a baseline time point and, for at least one of the multiple time periods, determining a new orientation signal from the axis signals. The method may further include determining an orientation change metric based on a difference between the new orientation signal and the baseline orientation signal. The method may include determining that the orientation change metric meets an orientation change threshold. The method may include detecting the dislodgement of the IMD in response to the metrics determined for at least the plurality of the multiple time periods meeting the dislodgement threshold and the orientation change metric meeting the orientation change threshold.

Example 23. The method of example 22 further including determining the orientation change metric based on the difference between the new orientation signal and the baseline orientation signal by determining at least one of a difference between an average amplitude of the new orientation signal and an average amplitude of the baseline orientation signal or a difference between an angle defined by an average amplitude of the orientation signal and an angle defined by an average amplitude of the baseline orientation signal.

Example 24. The method of example 22 further including receiving a second device orientation signal at the baseline time point and for the at least one of the multiple time periods. The method may include determining a baseline IMD to second device orientation metric from the second device orientation signal received at the baseline time point and the baseline orientation signal. The method may include, for the at least one of the multiple time periods, determining a new IMD to second device orientation metric from the second device orientation signal received for the at least one of the multiple time periods and the new orientation signal determined for the at least one of the multiple time periods. The method may include detecting an IMD orientation change based on the baseline IMD to second device orientation metric and the new IMD to second device orientation metric. The method may include detecting the dislodgement of the IMD in response to the metrics determined for at least the plurality of the multiple time periods meeting the dislodgement threshold and the detected IMD orientation change.

Example 25. The method of any of examples 16-24 further including detecting a sign change in at least one of the axis signals from a baseline time point to at least one of the multiple time periods and detecting the dislodgement of the IMD in response to the metrics determined for at least the plurality of the multiple time periods meeting the dislodgement threshold and the detected sign change.

Example 26. The method of any of examples 16-25 further comprising determining a long term trend value of the metric and determining the dislodgement threshold based on the long term trend value.

Example 27. The method of any of examples 16-26 further comprising, in response to detecting the dislodgement, adjusting at least one of a pacing control parameter used to control the pulse generator in generating the cardiac pacing pulses and/or a sensing control parameter used to control the sensing circuit in sensing the cardiac event signals.

Example 28. The method of any of examples 16-27 wherein determining the first signal from the axis signals may include determining a rectified axis signal from each of the axis signals sensed along the plurality of axes of the motion sensor and determining the first signal as a combination of the rectified axis signals.

Example 29. A non-transitory, computer-readable storage medium storing a set of instructions which, when executed by a control circuit of an IMD, cause the IMD to sense an axis signal along a plurality of axes of a motion sensor of the IMD and determine a signal from the axis signals sensed over multiple time periods. The multiple time periods may each extend over a plurality of cardiac cycles. For each of the multiple time periods, the instructions may further cause the IMD to determine a metric from the signal. The instructions may further cause the IMD to determine that the metrics determined for at least a plurality of the multiple time periods meet a dislodgement threshold. The instructions may cause the IMD to detect dislodgement of the IMD in response to the metrics determined for at least the plurality of multiple time periods meeting the dislodgement threshold. The instructions may further cause the IMD to transmit an IMD dislodgement alert in response to detecting the dislodgement.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. An implantable medical device (IMD) comprising:
a motion sensor configured to sense an axis signal along a plurality of axes of the motion sensor;
a sensing circuit configured to sense cardiac event signals attendant to myocardial depolarizations;
a pulse generator configured to generate cardiac pacing pulses; and
a control circuit in communication with the motion sensor, the sensing circuit and the pulse generator, the control circuit configured to:
determine a first signal from the axis signals sensed over multiple time periods that each extend over a plurality of cardiac cycles;
for each of the multiple time periods, determine a metric from the first signal;
determine that the metrics determined for at least a plurality of the multiple time periods meet a dislodgement threshold; and
detect dislodgement of the IMD in response to the metrics determined for at least the plurality of the multiple time periods meeting the dislodgement threshold; and
a communication circuit configured to transmit an IMD dislodgement alert in response to the control circuit detecting the dislodgement.

2. The IMD of claim 1 wherein the control circuit is further configured to:
identify cardiac cycles based on at least one of the cardiac event signals sensed by the sensing circuit or the cardiac pacing pulses generated by the pulse generator;
determine a peak amplitude of the first signal during each of a plurality of the identified cardiac cycles; and
determine the metric based on the peak amplitudes.

3. The IMD of claim 2 wherein the control circuit is further configured to:
detect an electrical indicator of dislodgement by determining at least one of:
a pacing impedance;
a pacing capture threshold;
a sensed cardiac event amplitude;
a loss of pacing capture;
evidence of undersensing of cardiac event signals; or
an increase in pacing burden; and
detect the dislodgement of the IMD in response to the metrics determined for at least the plurality of the multiple time periods meeting the dislodgement threshold and detecting the electrical indicator of dislodgement.

4. The IMD of claim 2 wherein the control circuit is further configured to determine the metric based on the peak amplitudes by:
for each of the peak amplitudes, determining a peak time of the peak amplitude relative to the corresponding identified cardiac cycle; and
determining the metric based on the peak times.

5. The IMD of claim 1 wherein the control circuit is further configured to:
filter the axis signals according to a first frequency range;
determine the first signal by determining a combination of the axis signals filtered according to the first frequency range;
filter the axis signals according to a second frequency range;
determine a second signal from a combination of the axis signals filtered according to the second frequency range; and
determine the metric by determining a difference between the first signal and the second signal.

6. The IMD of claim 5 wherein the control circuit is further configured to:
identify cardiac cycles based on at least one of the cardiac event signals sensed by the sensing circuit or the cardiac pacing pulses generated by the pulse generator;
determine a first peak amplitude of the first signal during each of a plurality of the identified cardiac cycles;
determine a second peak amplitude of the second signal during each of the plurality of the identified cardiac cycles; and
determine the difference between the first signal and the second signal based on the first peak amplitudes and the second peak amplitudes.

7. The IMD of claim 1 wherein the control circuit is further configured to:
determine a baseline orientation signal from the axis signals at a baseline time point;
for at least one of the multiple time periods, determine a new orientation signal from the axis signals;
determine an orientation change metric based on a difference between the new orientation signal and the baseline orientation signal;
determine that the orientation change metric meets an orientation change threshold; and
detect the dislodgement of the IMD in response to the metrics determined for at least the plurality of the multiple time periods meeting the dislodgement threshold and the orientation change metric meeting the orientation change threshold.

8. The IMD of claim 7 wherein the control circuit is further configured to determine the orientation change metric by determining at least one of:
a difference between an average amplitude of the new orientation signal and an average amplitude of the baseline orientation signal; or
a difference between an angle defined by an average amplitude of the orientation signal and an angle defined by an average amplitude of the baseline orientation signal.

9. The IMD of claim 7 wherein:
the communication circuit is further configured to receive a second device orientation signal at the baseline time point and the at least one of the multiple time periods; and
the control circuit is further configured to:
determine a baseline IMD to second device orientation metric from the second device orientation signal received at the baseline time point and the baseline orientation signal;
for at least one of the multiple time periods, determine a new IMD to second device orientation metric from the second device orientation signal received for the at least one of the multiple time periods and the new orientation signal received for the at least one of the multiple time periods;
detect an IMD orientation change based on the baseline IMD to second device orientation metric and the new IMD to second device orientation metric; and
detect the dislodgement of the IMD in response to the metrics determined for at least the plurality of the multiple time periods meeting the dislodgement threshold and the detected IMD orientation change.

10. The IMD of claim 1 wherein the control circuit is further configured to:
detect a sign change in at least one of the axis signals from a baseline time point to at least one of the multiple time periods; and
detect the dislodgement of the IMD in response to the metrics determined for at least the plurality of the multiple time periods meeting the dislodgement threshold and the detected sign change.

11. The IMD of claim 1 wherein the control circuit is further configured to:
determine a long term trend value of the metric;
determine the dislodgement threshold based on the long term trend value.

12. The IMD of claim 1 further comprising:
a housing enclosing the motion sensor, sensing circuit, pulse generator and the control circuit;
a plurality of electrodes on the housing; and
a fixation member extending from the housing for anchoring the IMD at an implant site.

13. The IMD of claim 12 wherein at least one electrode of the plurality of electrodes is the fixation member.

14. The IMD of claim 1 wherein, in response to detecting the dislodgement, the control circuit is configured to adjust at least one of:
a pacing control parameter used to control the pulse generator in generating the cardiac pacing pulses; or
a sensing control parameter used to control the sensing circuit in sensing the cardiac event signals.

15. The medical device of claim 1 wherein the control circuit is further configured to:
determine a rectified axis signal from each of the axis signals sensed along the plurality of axes of the motion sensor; and
determine the first signal as a combination of the rectified axis signals.

16. A method comprising:
sensing an axis signal along a plurality of axes of a motion sensor of an implantable medical device (IMD);
determining a first signal from the axis signals sensed over multiple time periods, the multiple time periods each extending over a plurality of cardiac cycles;
for each of the multiple time periods, determining a metric from the first signal;
determining that the metrics determined for at least a plurality of the multiple time periods meet a dislodgement threshold;
detecting dislodgement of the IMD in response to the metrics determined for at least the plurality of the multiple time periods meeting the dislodgement threshold; and
transmitting an IMD dislodgement alert in response to detecting the dislodgement.

17. The method of claim 16 further comprising:
filtering the axis signals according to a first frequency range;
determining the first signal by determining a combination of the axis signals filtered according to the first frequency range;
filtering the axis signals according to a second frequency range;
determining a second signal from a combination of the axis signals filtered according to the second frequency range; and
determining the metric by determining a difference between the first signal and the second signal.

18. The method of claim 16 further comprising:
determining a baseline orientation signal from the axis signals at a baseline time point;
for at least one of the multiple time periods, determining a new orientation signal from the axis signals;
determining an orientation change metric based on a difference between the new orientation signal and the baseline orientation signal;
determining that the orientation change metric meets an orientation change threshold; and
detecting the dislodgement of the IMD in response to the metrics determined for at least the plurality of the multiple time periods meeting the dislodgement threshold and the orientation change metric meeting the orientation change threshold.

19. The method of claim 18 further comprising:
receiving a second device orientation signal at the baseline time point and the at least one of the multiple time periods;
determining a baseline IMD to second device orientation metric from the second device orientation signal received at the baseline time point and the baseline orientation signal;
for the at least one of the multiple time periods, determining a new IMD to second device orientation metric from the second device orientation signal received for the at least one of the multiple time periods and the new orientation signal determined for the at least one of the multiple time periods;
detecting an IMD orientation change based on the baseline IMD to second device orientation metric and the new IMD to second device orientation metric; and
detecting the dislodgement of the IMD in response to the metrics determined for at least the plurality of the multiple time periods meeting the dislodgement threshold and the detected IMD orientation change.

20. The method of claim 16 further comprising:
determining a long term trend value of the metric; and
determining the dislodgement threshold based on the long term trend value.

21. A non-transitory, computer-readable storage medium storing a set of instructions which, when executed by a control circuit of an implantable medical device (IMD), cause the IMD to:
sense an axis signal along a plurality of axes of a motion sensor of the IMD;
determine a signal from the axis signals sensed over multiple time periods, the multiple time periods each extending over a plurality of cardiac cycles;
for each of the multiple time periods, determine a metric from the signal;
determine that the metrics determined for at least a plurality of the multiple time periods meet a dislodgement threshold;
detect dislodgement of the IMD in response to the metrics determined for at least the plurality of multiple time periods meeting the dislodgement threshold; and
transmit an IMD dislodgement alert in response to detecting the dislodgement.
